(19) United States
(12) Patent Application Publication
(10) Pub. No.: US 2024/0391964 A1
(43) Pub. Date: Nov. 28, 2024

(54) COMPOSITIONS AND METHODS FOR THE TREATMENT OF CANCER

(71) Applicant: The University of Western Australia, Nedlands (AU)

(72) Inventors: Pilar Blancaort, East Fremantle (AU); Ciara DUFFY, Nedlands (AU)

(73) Assignee: The University of Western Australia, Nedlands (AU)

(21) Appl. No.: 18/249,934

(22) PCT Filed: Jul. 16, 2021
(86) PCT No.: PCT/AU2021/050770

§ 371 Date: Apr. 20, 2023

(30) Foreign Application Priority Data
Jul. 17, 2020 (AU) 2020902478

(51) Int. Cl. C07K 14/435; A61K 31/337; A61K 33/243; A61K 38/17; A61P 35/00

(52) U.S. Cl. CPC C07K 14/43572; A61K 31/337; A61K 33/243; A61K 38/1767; A61P 35/00; C07K 2319/035

(57) ABSTRACT

The present invention relates to the field of compositions and methods for the treatment of cancer. In particular, the invention relates to novel melittin fusion peptides including an RGD-motif containing peptide that binds to the integrins αvβ6 and αvβ3. Preferably the RGD-motif containing fusion peptide can reduce viability of cancer cells, including in particular, TNBC and HER2-enriched breast cancer cells, and ovarian cancer cells.

### CROSS-REFERENCE TO RELATED APPLICATIONS

This application is a 35 U.S.C. 371 national stage of International Application No. PCT/AU2021/050770, filed Jul. 16, 2021 which claims the priority of Australia Application No. 2020902478, filed Jul. 17, 2020.

### SEQUENCE LISTING SUBMISSION VIA EFS-WEB

A computer readable text file, entitled “090415-5002-US Sequence Listing” created on May 30, 2024, with a file size of about 3,035 bytes, contains the sequence listing for this application and is hereby incorporated by reference in its entirety.

### TECHNICAL FIELD

The present invention relates to the field of compositions and methods for the treatment of cancer. In particular, the present invention relates to melittin fusion peptides and the use of melittin fusion peptides to treat cancers.

### BACKGROUND ART

The European honeybee (Apis mellifera) has been the source of a number of products used medicinally by humans such as honey, propolis and venom for thousands of years. Bee venom, particularly honeybee venom, has been shown to have anticancer activity. However, the molecular determinants of the anticancer selectivity of bee venoms remain poorly understood.

One of the components of honeybee venom is melittin, which comprises about half of honeybee venom by dry weight. Melittin is a positively charged, amphipathic 26 amino acid peptide of SEQ ID NO: 1 that associates with the phospholipids of the membrane bilayer, causing cell death by forming transmembrane toroidal pores. These pores of approximately 4.4 nm in diameter may also enable the internalisation of additional small molecules with cytotoxic activities.

Both honeybee venom and melittin have demonstrated anti-tumour effects in melanoma, non-small-cell lung cancer, glioblastoma, leukemia, hepatocellular carcinoma, ovarian, cervical, and pancreatic cancers, and have demonstrated higher killing potency in cancer cells compared to non-transformed cells. Additive and synergistic anticancer effects between honeybee venom and cisplatin have also been reported in cervical and laryngeal malignancies, and with docetaxel in lung cancer cells.

In breast cancer, honeybee venom reduced breast cancer lung metastasis, inhibited tumour growth, and prolonged survival in spontaneous mammary carcinoma murine tumours. Honeybee venom and melittin induced apoptosis in luminal MCF7 cells, and reduced cell viability and migration in triple negative MDA-MB-231 cells.

Most of the anti-neoplastic activity of honeybee venom has been attributed to melittin through inhibition of the PI3K/Akt/mTOR axis in breast cancer, MAPK in hepatocyte and melanoma cells, JAK2/STAT3 in ovarian cancer, and NFκB signaling pathways in lung carcinoma cells.

However, the precise effect of melittin across different breast cancer subtypes is still not well understood, even though breast cancer is the most common cancer in women worldwide. In particular, triple negative breast cancer (TNBC), which lacks the expression of hormone receptors and HER2, and HER2-enriched breast cancer, are aggressive and associated with poor outcomes in patients.

Approximately 50% of TNBCs overexpress epidermal growth factor receptor (EGFR), and HER2-enriched tumours overexpress HER2, another receptor tyrosine kinase (RTK) of the same family. RTKs confer oncogenic signaling often dependent on the PI3K/Akt pathway downstream. Blocking EGFR signaling in TNBC has demonstrated limited clinical efficacy in early phase clinical trials due to a lack of dependence on the EGFR pathway and the importance of collateral pathways. Although HER2-targeted therapies have dramatically improved median survival in the metastatic setting, resistance is almost inevitable over the longer term for this subtype.

The precise effect of melittin is also poorly understood in relation to ovarian cancer. Ovarian cancer (OC) is a type of gynecological cancer that originate in the reproductive system. OC is the deadliest gynecological cancer, associated with a very poor 5-year survival of 46%, with 1,068 estimated deaths and 1,532 new diagnoses in Australia in 2020. Whist survival rates from other solid cancers have improved in last decades, the rates survival from OC have remained unchanged since the 1980s. This is associated to several factors, including the non-specific symptoms of the disease, the lack of effective screening strategies, and consequently, most (75-80%) of these cancers are diagnosed at advanced stages. The vast majority (˜90%) of OCs have an epithelial origin, and among these, high grade serious ovarian cancers (HGSOCs) are highly proliferative and aggressive, and thought to originate from the fallopian tube. These cancers are characterized by P53 mutations and by high genomic instability as a consequence of defects of pathways involving homologous recombination (HR) DNA repair genes. These include mutations in the tumour suppressors BRCA1, BRCA2 which confer high risk to develop HGSOC. The BRCA1/2 mutations (germline and somatic) occur in 20% of the patients, while DNA hypermethylation and epigenetic silencing in the BRCA1 gene is observed in 15% of cases.

Ovarian cancer is typically associated with a poor prognosis for patients, so there is a need for improved treatments. The effects of melittin have been reported in a limited number of ovarian cancer cell lines, being PA-1, SKOV3 and OVCAR3.

There is therefore a need for the development of more effective and selective therapeutic strategies for the treatment of these cancers. As with all cancer therapies, these therapies should be selective for the relevant cancer cells.

The present invention provides a novel fusion peptide, that can reduce cell viability in cancer cells, including in particular, TNBC and HER2-enriched breast cancer cells, and ovarian cancer cells.

The previous discussion of the background art is intended to facilitate an understanding of the present invention only. The discussion is not an acknowledgement or admission that any of the material referred to is or was part of the common general knowledge as at the priority date of the application.

### SUMMARY OF INVENTION

In an aspect, the present invention provides a novel fusion peptide, that can reduce cell viability in cancer cells, including in particular, TNBC and HER2-enriched breast cancer cells, and ovarian cancer cells. References to “fusion peptides” and “melittin fusion peptides” of the invention, throughout this specification, should be read as a reference to a fusion peptide comprising (1) SEQ ID NO: 1, or a variant thereof, and (2) an RGD-motif containing peptide.

In a first aspect, the invention provides a fusion peptide comprising: (1) SEQ ID NO: 1, or a variant thereof, and (2) an RGD-motif containing peptide. Preferably, the fusion peptide comprises SEQ ID NO: 1, or a variant thereof, bound to an RGD-motif containing peptide, wherein the fusion peptide binds to the integrins αvβ6 and αvβ3. The integrins αvβ6 and/or αvβ3 are commonly overexpressed in TNBC and HER2-enriched breast cancer cells, as well as ovarian cancer cells. The invention also provides variants of the fusion peptides described herein.

Preferably, the RGD-motif containing peptide comprises SEQ ID NO: 2 or a variant thereof. In a most preferred embodiment, the fusion peptide comprises SEQ ID NO: 3, or a variant thereof.

In a second aspect, the invention provides a fusion peptide of SEQ ID NO: 3 or a variant thereof.

In a third aspect, the invention provides a pharmaceutical composition comprising a fusion peptide of the invention. Preferably, the pharmaceutical composition additionally comprises a pharmaceutically acceptable diluent, carrier, solubilizer, emulsifier, preservative and/or adjuvant. Preferably, the pharmaceutical composition may additionally comprise a chemotherapeutic agent. Preferably, the chemotherapeutic agent is selected from docetaxel or cisplatin.

In a fourth aspect, the invention provides a method of treating cancer in a subject comprising administering to the said subject a therapeutically effective amount of a fusion peptide of the invention.

Preferably, the cancer is selected from the group of HER2-enriched breast cancer, triple negative breast cancer or ovarian cancer.

Preferably, the method of treating cancer additionally comprises the step of administering to said subject a chemotherapeutic agent. Preferably, the chemotherapeutic agent is selected from one or more of pertuzumab, trastuzumab, docetaxel or cisplatin.

In another embodiment of the fourth aspect, the method of treating cancer additionally comprises administering to said subject an anticancer therapy. Preferably, the anticancer therapy is omomyc or a Cas9-gRNA complex or variant thereof.

In a fifth aspect, the invention provides a fusion peptide of the invention for use in a method of treating cancer in a subject in need thereof.

In a sixth aspect, the invention provides the use of a fusion peptide of the invention in the manufacture of a medicament for treating cancer in a subject in need thereof.

In a seventh aspect, the invention provides a method of treating triple negative breast cancer, HER2 enriched breast cancer or ovarian cancer in a subject comprising the steps of: (1) administering to said subject a therapeutically effective amount of a combination therapy of melittin or fusion peptide; and (2) administering a therapeutically effective amount of a chemotherapeutic agent. Preferably, the chemotherapeutic agent is selected from pertuzumab, trastuzumab, docetaxel, and/or cisplatin.

In another embodiment of the seventh aspect, the invention provides a method of treating triple negative breast cancer, HER2 enriched breast cancer or ovarian cancer in a subject comprising administering to said subject a therapeutically effective amount of a combination therapy of melittin or melittin fusion peptide and an anticancer therapy. Preferably, the anticancer therapy is omomyc or a Cas9-gRNA complex or variant thereof.

In an eighth aspect, the invention provides a method of reducing tumour cell viability in a subject, said method comprising the step of administration of a therapeutically effective amount of a melittin fusion peptide of the invention. Preferably the melittin fusion peptide comprises SEQ ID NO: 3. Preferably, the method further comprises the step of administering to said subject a chemotherapeutic agent. Preferably, the chemotherapeutic agent is selected from one or more of pertuzumab, trastuzumab, docetaxel, or cisplatin.

In a ninth aspect, the invention provides a kit for treating cancer in a subject comprising a melittin fusion peptide of the invention together with instructions as to how to use that fusion peptide in accordance with this invention. Preferably, the kit additionally comprises a chemotherapeutic agent. Preferably, the chemotherapeutic agent is selected from pertuzumab, trastuzumab, docetaxel, or cisplatin.

In a tenth aspect, the invention provides a nucleic acid encoding a melittin fusion peptide. Preferably, the nucleic acid encodes a melittin fusion peptide of SEQ ID NO: 3, of SEQ ID NO: 7.

In an eleventh aspect, the invention provides a method of inhibiting PD-L1 expression in a subject suffering from cancer comprising the step of administering melittin or a fusion peptide in a therapeutically effective amount to the subject. Preferably, the method additionally comprises the step of administering a chemotherapeutic agent. More preferably, the chemotherapeutic agent is selected from one or more of pertuzumab, trastuzumab, docetaxel or cisplatin. Preferably, the subject is suffering from triple negative breast cancer, HER2-enriched breast cancer or ovarian cancer.

In a twelfth aspect, the invention provides the use of the fusion peptide of the invention in the manufacture of a medicament for inhibiting PD-L1 expression in a subject suffering from cancer.

In a thirteenth aspect, the invention provides an isolated monoclonal antibody that specifically binds to the peptides of SEQ ID NO:1 and SEQ ID NO: 3.

In a fourteenth aspect, the invention provides a method of facilitating the cell penetration of an anticancer therapy in a subject suffering from cancer, comprising the step of administering a therapeutically effective amount of a combination therapy comprising administering: (1) of melittin or a or a fusion peptide; and (2) an anticancer therapy to the subject. Preferably, the anticancer therapy is omomyc or a Cas9-gRNA complex or variant thereof.

In a fifteenth aspect, the invention provides a fusion peptide comprising (1) SEQ ID NO: 1, or a variant thereof, and (2) an RGD-motif containing peptide, wherein the fusion peptide binds to the integrins αvβ6 and αvβ3.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings.

FIG. 1 presents results of tumour viability assays for cell lines exposed to bee venom and melittin. FIG. 1A presents cell viability assays of a panel of human normal and breast cancer cell lines treated with Australian honeybee venom (left) or melittin (right), with the IC50 values presented in FIG. 1B. FIG. 1C presents cell viability dose-response curves of a panel of murine non-transformed (NIH/3T3), basal-like breast cancer (BRCA⁻ B.15) and claudin-low breast cancer (p53⁻ T11) cell lines treated with Australian honeybee venom (left) or melittin (right). FIG. 1D presents the IC50 values for each cell type exposed to honeybee venom and melittin.

FIG. 2: FIG. 2A presents cell viability dose-response curves of human dermal fibroblasts (HDFa) and breast cancer cell lines (SUM159 and SKBR3) treated with venom from populations of honeybees in Ireland (left) and England (right). FIG. 2B presents cell viability dose-response curves of human dermal fibroblasts (HDFa) and breast cancer cell lines (SUM159 and SKBR3) treated with venom from populations of England worker (left) and queen (right) bumblebees.

FIG. 3: FIG. 3A presents absorbance (405 nm) of aqueous solutions of melittin and bee venom assessed by ELISA with the anti-melittin antibody and IgG control. FIG. 3B presents cell viability assays in HDFa and SUM159 after blocking melittin using the anti-melittin antibody with honeybee venom (top) and melittin (bottom). Data are represented as mean±SEM.

FIG. 4 presents results of the mechanism and kinetics of cell death for TNBC (SUM159), HER2-enriched (SKBR3), and normal (HDFa) cells treated with honeybee venom or melittin. FIG. 4A presents a Western blot for the detection of cleaved caspase-3 (CL-csp-3) in SUM159 cells treated with vehicle (1), honeybee venom (2-3) and melittin (4-5) for 18 and 24 hours. FIG. 4B presents flow cytometry analysis of SUM159 cells treated with the IC50 of honeybee venom (5.58 ng/μL) and the IC50 of melittin (4.24 ng/μL) for one hour. FIG. 4C presents cell viability assays of human dermal fibroblasts (HDFa) and breast cancer cells (SUM159 and SKBR3) treated with honeybee venom (left) or melittin (right) over one hour. FIG. 4D presents live cell confocal microscopy of SKBR3 cells treated with the IC50 of honeybee venom (5.77 ng/μL) over one hour, with time in minutes' post-treatment. FIG. 4E presents scanning electron microscopy of SUM159 cells treated with vehicle, the IC50 of honeybee venom (5.58 ng/μL) or the IC50 of melittin (4.24 ng/μL) over one hour, with two representative images shown for each treatment group. Scale bars represent 200 nm, 1 μm or 10 μm as indicated. Data are represented as mean±SEM.

FIG. 5 presents the lack of anticancer activity of DEDE-melittin, and the breast cancer cell selectivity of the melittin fusion peptide RGD1-melittin. FIG. 5A presents cell viability assays of TNBC (SUM159) and HER2-enriched (SKBR3) cells treated with DEDE-melittin for 24 hours. FIG. 5B presents cell viability assays of T11 cells treated with melittin, RGD1-melittin, SV40-melittin, TAT-melittin and DEDE-melittin. FIG. 5C presents cell viability assays of human dermal fibroblasts (HDFa) and SUM159 treated with melittin (top) and RGD1-melittin (bottom) for 24 hours.

FIG. 6 presents the lack of anticancer activity of DEDE-melittin, and the breast cancer cell selectivity of the melittin fusion peptide RGD1-melittin. FIG. 6A presents a Western blot for the detection of cleaved caspase-3 (CL-csp-3) in cell lysates from SUM159 cells treated with either vehicle, melittin, DEDE-melittin or RGD1-melittin for 24 hours. FIG. 6B presents absorbance (405 nm) of aqueous solutions of melittin, RGD1-melittin, DEDE-melittin and SV40-melittin subjected to an ELISA with the anti-melittin antibody. FIG. 6C presents the amino acid sequence and top predicted 3D model of melittin, RGD1-melittin, DEDE-melittin and SV40-melittin.

FIG. 7 presents immunofluorescence images of SUM159 treated with vehicle, honeybee venom, melittin, RGD1-melittin or DEDE-melittin for 30 minutes.

FIG. 8 presents the phosphorylation kinetics of EGFR, HER2 and downstream MAPK and Akt pathways after treatment with honeybee venom and melittin in (A) SKBR3 and (B) SUM159, and melittin in (C) MDA-MB-231 breast cancer cells, assessed by immunoblotting.

FIG. 9 presents a Western blot for the detection of JAK/STAT pathway inhibitors in SUM159 cells treated with vehicle, honeybee venom or melittin for 60 minutes, with and without 20 ng/ml EGF for 5 minutes. α-Tubulin was used as the loading control.

FIG. 10 presents the kinetic analysis of (A) TAMRA-EGF, (B) FITC-melittin, (C) FITC-DEDE-melittin, and (D) FITC-EN1-mutant interaction with NanoLuc-EGFR by bioluminescence resonance energy transfer (BRET) in HEK293FT cells.

FIG. 11 presents the saturation binding analysis of increasing concentrations of (A) TAMRA-EGF, (B) FITC-melittin and (C) FITC-DEDE-melittin in HEK293FT cells transfected with NanoLuc-EGFR in the presence or absence of unlabeled EGF (1 μM).

FIG. 12 presents the results of in vitro assays of the combination of honeybee venom or melittin with docetaxel or cisplatin administered to TNBC cells. FIG. 12A presents cell viability assays of T11 breast cancer cells treated with honeybee venom or melittin alone and in combination with docetaxel or cisplatin for 24 hours. Representative plots of the combination treatments are presented as mean±SEM. FIG. 12B presents Combination Index graphs obtained for different fractions of cells affected in each combination treatment in 12A, calculated using CompuSyn software. A synergistic drug combination is indicated by CI<1.

FIG. 13 presents the results of in vivo assays of the combination of docetaxel administered to mice. FIG. 13A presents tumour volumes of mice treated intratumorally with vehicle, 5 mg/Kg melittin, 7 mg/Kg docetaxel, or 5 mg/Kg melittin+7 mg/Kg docetaxel. Arrows indicate the treatment days. Corresponding scatter plots of relative change in tumour volumes at days 3, 7 and 9 are indicated. FIG. 13B presents representative bioluminescence imaging (BLI) images of T11-luciferase tumours in mice at days 4, 10, 12 and 14 post-inoculation of the cells.

FIG. 14: FIG. 14A presents the quantification of immunohistochemistry and immunofluorescence in tumour biopsies from mice extracted on day 14 post-T11 inoculation stained with anti-melittin, anti-Ki-67, anti-PD-L1, with TUNEL assay, and with Hoechst and H&E. FIG. 14B presents the quantification of immunohistochemistry in tumour biopsies from mice extracted on day 14 post-T11 cells inoculation, evaluating the effect of single or combined inhibition with melittin and docetaxel on the expression levels of EGFR and HER2 and reduction of p-EGFR (Tyr1068) and p-HER2 (Tyr1248).

FIG. 15 presents the results of cell viability assays of T11 breast cancer cells treated with melittin or RGD1-melittin alone and in combination with docetaxel for 48 hours. Representative plots of the combination treatments are presented as mean±SEM.

FIG. 16 presents the results of cell viability assays of T11 breast cancer cells treated with melittin or RGD1-melittin alone and in combination with omomyc for 24 hours. Representative plots of the combination treatments are presented as mean±SEM.

FIG. 17: FIG. 17A presents the results of cell viability assays of OVCAR3 and COV362 ovarian cancer cells treated with active RGD1-melittin and mutant RGD1-melittin (RGD1-DEDE melittin). FIG. 17B presents the results of cell viability assays of OVCAR3 and COV362 ovarian cancer cells with active melittin and mutant melittin (DEDE melittin).

FIG. 18: FIG. 18A presents immunofluorescence images of PD-L1 expression in T11, SUM159 and SKBR3 breast cancer cells treated with RGD1-melittin and mutant RGD1-melittin (DEDE-RGD1-melittin). FIG. 18B presents qRT-PCT analysis of PD-L1 expression levels in T11, SUM159 and SKBR3 treated with active RGD1-melittin and mutant RGD1-melittin peptides, or untreated (media only) for 24 h. Significance levels were determined relative to the untreated condition for each cell line, respectively, using the ordinary one-way ANOVA where *, **, *** and **** represent p<0.05, p<0.005, p<0.0005 and p<0.0001, respectively.

FIG. 19 presents the results of cell viability assays of T11, SUM159, OVCAR3, and SKBR3 breast and ovarian cancer cells treated with RGD1-melittin and docetaxel alone and in combination.

FIG. 20 presents the results of cell viability assays of T11, OVCAR3, and COV362 breast and ovarian cancer cells treated with RGD1-melittin and cisplatin alone and in combination.

### BRIEF DESCRIPTION OF THE SEQUENCE LISTINGS

The description refers to the following amino acid and nucleic acid sequences:

| SEQ ID NO: | Description | Sequence |
|---|---|---|
| SEQ ID NO: 1 | Melittin peptide | GIGAVLKVLTTGLPALISWIKRKRQQ |
| SEQ ID NO: 2 | RGD-motif | HGRGDLGRLKK |
| | containing peptide | |
| SEQ ID NO: 3 | Melittin fusion | HGRGDLGRLKKGIGAVLKVLTTGLPALISWIKRKR |
| | peptide (RGD1- | QQ |
| | melittin) | |
| SEQ ID NO: 4 | Positive sequence | KRKR |
| | in the C-terminal of | |
| | melittin | |
| SEQ ID NO: 5 | C-terminal | KKKRKV |
| | sequence from | |
| | SV40 | |
| SEQ ID NO: 6 | Negatively charged | GIGAVLKVLTTGLPALISWIDEDEQQ |
| | Melittin C-terminal | |
| | peptide (DEDE- | |
| | melittin) | |
| SEQ ID NO: 7 | Nucleic acid | CACGGCAGGGGCGACCTGGGCAGGCTGAAGA |
| | sequence for | AGGGCATCGGCGCCGTGCTGAAGGTGCTGAC |
| | RGD1-melittin | CACCGGCCTGCCCGCCCTGATCAGCTGGATCA |
| | (Homo Sapiens) | AGAGGAAGAGGCAGCAG |
| SEQ ID NO: 8 | Nucleic acid | CATGGCCGCGGCGATCTGGGCCGCCTGAAAAA |
| | sequence for | AGGCATTGGCGCGGTGCTGAAAGTGCTGACCA |
| | RGD1-melittin (E. | CCGGCCTGCCGGCGCTGATTAGCTGGATTAAA |
| | coli) | CGCAAACGCCAGCAG |
| SEQ ID NO: 9 | SV40-melittin | GIGAVLKVLTTGLPALISWIKKKRKV |
| SEQ ID NO: 10 | TAT-melittin | GIGAVLKVLTTGLPALISWIGRKKRRQRRRPQ |

### DETAILED DESCRIPTION OF INVENTION

The present invention is based on the discovery that certain melittin fusion peptides have enhanced selectivity for certain subtypes of cancer cells. Preferably, the melittin fusion peptides have enhanced selectivity for specific subtypes of breast cancer or ovarian cancer.

### A. General

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (e.g. dosage, concentration etc.). A range of values will be understood to include all values within the range, including the values defining the range, and values adjacent to the range which lead to the same or substantially the same outcome as the values immediately adjacent to that value which defines the boundary to the range. Accordingly, unless indicated to the contrary, the numerical parameters set forth in the specification and claims are approximations that may vary depending upon the desired properties sought to be obtained by the present invention. Hence “about 80%” means “about 80%” and also “80%”. At the very least, each numerical parameter should be construed in light of the number of significant digits and ordinary rounding approaches.

It is to be understood that both the foregoing general description and the following detailed description are exemplary and explanatory only and are not restrictive of the invention as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

The terms “polypeptide”, “peptide” or “protein” means a macromolecule having the amino acid sequence of a native protein, that is, a protein produced by a naturally-occurring and non-recombinant cell; or it is produced by a genetically-engineered or recombinant cell, and comprise molecules having the amino acid sequence of the native protein, or molecules having deletions from, additions to, and/or substitutions of one or more amino acids of the native sequence. The terms “polypeptide”, “peptide” and “protein” specifically encompass melittin and melittin fusion peptides or sequences that have deletions from, additions to, and/or substitutions of one or more amino acid of the melittin or melittin fusion peptide without adversely affecting the functionality of the peptide.

A “variant” of a polypeptide (e.g. melittin fusion peptide) comprises an amino acid sequence wherein one or more amino acid residues are inserted into, deleted from and/or substituted into the amino acid sequence relative to another polypeptide sequence, but which retains the biological activity of the original polypeptide sequence by reference to assays known in the art. Variants include fusion proteins. A “variant” of a melittin fusion peptide comprises an amino acid sequence wherein one or more amino acid residues are inserted into, deleted from and/or substituted into the amino acid sequence relative to the melittin fusion peptide, which retains melittin fusion peptide like biological activity, which can be measured by assays known in the art, such as cell viability assays.

The term “therapeutically effective amount” refers to the amount of melittin or a melittin fusion peptide determined to produce a therapeutic response in a mammal, including a human. Such therapeutically effective amounts are readily ascertained by one of ordinary skill in the art.

The terms “patient” and “subject” are used interchangeably and include human and non-human animal subjects with formally diagnosed disorders, those without formally recognized disorders, those receiving medical attention and those at risk of developing the disorders.

The terms “treat” and “treatment” includes therapeutic treatments, prophylactic treatments, and applications in which one reduces the risk that a subject will develop a disorder or other risk factor. Treatment does not require the complete curing of a disorder and encompasses embodiments in which one reduces symptoms, underlying risk factors or delays progression of the disorder.

The term “cancer” as used herein, refers to an abnormal growth of cells that tend to proliferate in an uncontrolled way and, in some cases, to metastasize (spread). Types of cancer include, but are not limited to, solid tumours (such as those of the bladder, bowel, brain, breast, endometrium, heart, kidney, lung, liver, uterus, lymphatic tissue [lymphoma], ovary, pancreas or other endocrine organ [thyroid], prostate, skin [melanoma or basal cell cancer]), or haematological tumours (such as the leukemias and lymphomas) at any stage of the disease with or without metastases.

The term “triple negative breast cancer” or “TNBC” as used herein refers to a type of breast cancer where oestrogen, progesterone and HER2 receptors are not expressed by the tumour cells.

The term “HER2-enriched breast cancer” as used herein refers to a type of breast cancer where human epidermal growth factor receptor 2 (HER2) is overexpressed by the tumour cells.

The term “ovarian cancer” as used herein refers to cancer that forms in or on an ovary.

The term “binds” or “interacts” has the meaning known in the art. The term “binding” in connection with the interaction between a one compound or molecule and another compound or molecule, such as a target and a potential binding compound, indicates that the potential binding compound associates with the target to a statistically significant degree as compared to association with proteins generally. Thus, the term “specific binding” refers to binding between two molecules or compounds that is statistically significantly higher than non-specific binding to another molecule. Preferably a binding compound interacts with a specified target with a dissociation constant (k.sub.d) of 1 mM or less, for example 0.1-100 nM. A binding compound can bind with “low affinity”, “very low affinity”, “extremely low affinity”, “moderate affinity”, “moderately high affinity”, or “high affinity”. In the context of compounds binding to a target, the term “greater affinity” indicates that the compound binds more tightly than a reference compound, or than the same compound in a reference condition, i.e., with a lower dissociation constant. In particular embodiments, the greater affinity is at least 2, 3, 4, 5, 8, 10, 50, 100, 200, 400, 500, 1000, or 10,000-fold greater affinity. Also in the context of compounds binding to a biomolecular target, the term “greater specificity” indicates that a compound binds to a specified target to a greater extent than to another biomolecule or biomolecules that may be present under relevant binding conditions, where binding to such other biomolecules produces a different biological activity than binding to the specified target. Typically, the specificity is with reference to a limited set of other biomolecules. In particular embodiments, the greater specificity is at least 2, 3, 4, 5, 8, 10, 50, 100, 200, 400, 500, or 1000-fold greater specificity. Binding can be measured by various assays known in the art, including mobility shift experiments, surface plasmon resonance, fluorescence polarization, fluorescence resonance energy transfer, isothermal titration calorimetry (ITC) and microscale thermophoresis (MST) methods.

As used herein, “melittin” refers to the 26 amino acid length peptide of SEQ ID NO: 1 that is derived from honeybee venom.

The term “melittin fusion peptide” refers to melittin of SEQ ID NO: 1 or a variant thereof fused with another peptide, protein or fragment thereof.

The term “isolated peptide” referred means that a subject protein (1) is free of at least some other proteins with which it would normally be found, (2) is essentially free of other proteins from the same source, e.g., from the same species, (3) is expressed by a cell from a different species, (4) has been separated from at least about 50 percent of polynucleotides, lipids, carbohydrates, or other materials with which it is associated in nature, (5) is operably associated (by covalent or noncovalent interaction) with a polypeptide with which it is not associated in nature, or (6) does not occur in nature. Typically, an “isolated peptide” constitutes at least about 5%, at least about 10%, at least about 25%, or at least about 50% of a given sample. Genomic DNA, cDNA, mRNA or other RNA, of synthetic origin, or any combination thereof can encode such an isolated protein. Preferably, the isolated protein is substantially free from proteins or polypeptides or other contaminants that are found in its natural environment that would interfere with its therapeutic, diagnostic, prophylactic, research or other use.

The term “chemotherapeutic agent” as used herein means a chemical compound useful in the treatment of cancer. Examples of chemotherapeutic agents include alkylating agents, such as, for example, temozolomide, the imidazotetrazine derivative of the alkylating agent dacarbazine. Additional examples of chemotherapeutics agents include, e.g., paclitaxel or topotecan or pegylated liposomal doxorubicin (PLD). Other examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin; bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBT TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall [see, e.g., Agnew, Chem. Intl. Ed. Engl., 33:183-186 (1994)]; dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins (bleomycin A2), cactinomycin, carabicin, caminomycin, carzinophilin, daunorubicin, chromomycinis, dactinomycin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide, glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK@ polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® docetaxel (Rhone-Poulene Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylomithine (DMFO); retinoids such as retinoic acid; capecitabine; combretastatin; leucovorin (LV); oxaliplatin, including the oxaliplatin treatment regimen (FOLFOX); lapatinib (Tykerb®); pertuzumab and trastuzumab, inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above.

An “anticancer therapy” is any compound, composition or treatment that prevents or delays the growth and/or metastasis of cancer cells. Such anticancer therapies include but are not limited to chemotherapeutic agents, radiation, gene therapy, hormone therapy, immunotherapy, polypeptides and antisense oligonucleotide therapy.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs. The term “active agent” may mean one active agent or may encompass two or more active agents.

### B. Melittin Fusion Peptides

In an aspect, the invention provides a melittin fusion peptide comprising melittin of SEQ ID NO: 1 bound to an RGD-motif containing peptide. In another aspect, the invention provides a melittin fusion peptide comprising (1) melittin of SEQ ID NO: 1 and (2) an RGD-motif containing peptide. Preferably, the melittin fusion peptide binds to the integrins αvβ6 and αvβ3. Preferably the RGD-motif containing peptide comprises SEQ ID NO: 2. In a most preferred embodiment, the invention provides a melittin fusion peptide of SEQ ID NO: 3, or a variant thereof.

### (a) Melittin

Melittin is a polypeptide component of honeybee venom. Melittin is known to reduce tumour cell viability. Melittin is thought to reduce tumour cell viability by binding to the plasma membrane, inducing subsequent pore formation, which leads to the leaking of cellular ions and molecules, an enhancement of cell permeability and ultimately cell lysis.

Melittin may also have an effect on apoptosis and necrosis of cells. The specific mechanism of action of melittin in reducing tumour cell viability can depend on the type of tumour cell. In respect of triple negative breast cancer expressing EGF receptors and HER2, melittin can suppress ligand induced phosphorylation of EGFR and HER2, dynamically modulating downstream signaling pathways in breast cancer cells.

Immune checkpoint protein Programmed Death Ligand-1 (PD-L1) binds to the immune checkpoint protein PD-1 and can inhibit the activation and immune recognition of T-cells that would otherwise be involved in the natural immune response against tumour cells. The inhibition of PD-L1 results in the activation of T cells. It has also been shown that in certain cancers, the administration of certain chemotherapeutic agents can increase the expression of PD-L1, resulting in reduced immune response to the tumour cell, and reduced overall efficacy of the treatment. For example, in HER2-positive breast cancer cells co-cultured with human peripheral blood mononuclear cells and in a mouse model, trastuzumab anti-HER2 therapy was shown to result in the upregulation of PD-L1 (B. K. R. et al. Cancer Lett. 430, 47-56 (2018)). Similarly, ovarian cancer cells have been shown to upregulate PD-L1 expression in response to chemotherapy exposure (Grabosch, S., Zeng, F., Zhang, L. et al. PD-L1 biology in response to chemotherapy in vitro and in vivo in ovarian cancer. j. immunotherapy cancer 3, P302 (2015)).

In some embodiments of this invention, melittin may reduce the levels of PD-L1 immune check-point protein. Melittin therefore may decrease the immune-suppressive effects of the tumour microenvironment, which are prevalent in cancers such as triple negative breast cancer in the presence of chemotherapy. In some embodiments, melittin may attenuate the increased expression of PD-L1 caused by chemotherapeutic agents, such as trastuzumab. Melittin has been shown to reduce the tumour promoting M2-like tumour-associated macrophage population in the tumour microenvironment in a lung carcinoma model.

In some embodiments, EGFR and HER2 signaling in breast cancer cells modulates PD-L1 expression in tumour cells. Immunohistochemical studies indicate that PD-L1 has the highest expression in TNBC tumours, followed by HER2-enriched tumours, and PD-L1 expression is associated with poor survival. In other embodiments, in basal-like breast cancers, the absence of the protein ALIX may correlate with EGFR activation, impairing exosome biogenesis. PD-L1 is secreted via exosomes in an ALIX-dependent manner such that exosome impairment increases PD-L1 on the cell membrane. ALIX downregulation promotes tumour survival through enhancement of EGFR activation, and through PD-L1 membrane accumulation, leading to immunosuppression.

The C-terminus of melittin forms a positively charged α-helix. The positively charged alpha helix mediates binding to the negatively charged plasma membrane, then resulting in the formation of pores in the cell plasma membrane. In an embodiment, the positive K₂₁RKR₂₄ sequence of the C-terminus of melittin mediates binding to the negatively charged plasma membrane and is therefore essential to its function in disrupting the cell plasma membrane.

### (b) RGD-Motif Containing Peptide

Selectivity of melittin to tumour cell types is important for enhancing the ability of melittin to target tumour cells rather than exerting its effects on healthy cells. In an embodiment, the selectivity of melittin to specific tumour cells is enhanced by fusing melittin to another peptide. Preferably, the melittin is fused with a peptide containing an RGD peptide motif.

An “RGD” motif is a tri-peptide motif consisting of the amino acids arginine, glycine and aspartate. RGD motifs are present on a number of different extracellular proteins. Many such proteins are recognised by some members of the integrin protein family via the RGD motif. Of the 24 human integrin subtypes known, eight integrin dimers, i.e., αvβ1, αvβ3, αvβ5, αvβ6, αvβ8, α5β1, α8β1, and αIIbβ3, recognize the tripeptide RGD-motif within extracellular matrix proteins.

Certain proteins containing RGD motifs are selective for particular members of the integrin protein family. For example, the ligand TGF-β3, which is known to regulate molecules involved in cellular adhesion and extracellular matrix formation during the process of palate development, contains an RGD-motif and is known to interact with the integrins αvβ6 and αvβ3 via the RGD-motif. There are also a number of other molecules known to bind to members of the integrin protein family. For example, TGF-β1, the foot-and-mouth-disease virus (FMDV) and fibronectin are known to bind to αvβ6.

For RGD-motif containing peptides, there are other peptide options: see Dong, X., Hudson, N. E., Lu, C. & Springer, T. A. Structural determinants of integrin β-subunit specificity for latent TGF-β. Nat. Struct. Mol. Biol. 21, 1091-1096 (2014); Sorolla, A. et al. Triple-hit therapeutic approach for triple negative breast cancers using docetaxel nanoparticles, EN1-iPeps and RGD peptides. Nanomedicine Nanotechnology, Biol. Med. 102003 (2019), which are incorporated in their totality by reference.

Certain members of the integrin protein family are overexpressed on the plasma membrane of specific tumour cell types and associated vasculature. Therefore, peptides containing an RGD-motif can be useful for targeting tumour cell types that overexpress the integrins to which the RGD-containing peptides bind.

Integrins αvβ6 and αvβ3 are over-expressed on the plasma membrane of certain breast cancer cells and tumour associated vasculature. In particular, αvβ6 and αvβ3 are overexpressed on the surface of triple negative breast cancer and HER2-enriched breast cancer cells. Therefore, peptides that selectively bind integrins αvβ6 and αvβ3 can be selective towards triple negative breast cancer and HER2-enriched breast cancer cells. Integrin αvβ6 is over-expressed on the plasma membrane of ovarian cancer cells (see N Ahmed et. al. Carcinogenesis vol. 23 no. 2 pp. 237-244, 2002). In a preferred embodiment, the RGD motif of SEQ ID NO: 2 (RGD1) has the best attraction, that is binding affinity, to αvβ6 integrins making it potent to breast cancer and ovarian cancer cells. In an embodiment of the invention, RGD1 also binds, with lower affinity, to αvβ3 integrin.

Accordingly, in aspect, the invention provides for a melittin fusion peptide wherein the melittin of SEQ ID NO: 1 is fused with a peptide containing an RGD peptide motif, which recognises integrins αvβ6 and αvβ3. Preferably, the melittin fusion peptide of the invention has enhanced selectivity for HER2-enriched, triple negative breast cancer cell subtypes and/or ovarian cancer. Most preferably, the melittin fusion peptide retains the anti-tumoral activity of melittin, and exerts substantially the same, or superior, effects on tumour cell viability as melittin. In some embodiments, the melittin fusion peptide experts substantially the same, or superior, effects on PD-L1 expression in tumour cells.

Preferably, the sequence of the RGD motif containing peptide is derived from TGF-β3. More preferably, the RGD motif containing peptide has the sequence HGRGDLGRLKK (SEQ ID NO: 2) (RGD1). Preferably, RGD1 interacts with both αvβ6 and αvβ3 integrins, with particularly high affinity for αvβ6 integrin.

In a most preferred embodiment, melittin is fused with the RGD motif of TGF-β3 of SEQ ID NO: 2 at the N-terminus, and has the following sequence:

(SEQ ID NO: 3)
HGRGDLGRLKKGIGAVLKVLTTGLPALISWIKRKRQQ

In an aspect, the fusion peptide of the invention is an isolated peptide.

### (c) Fusion of Melittin to RGD Motif Containing Peptide

In an aspect, the melittin fusion peptides of the invention are bi-functional in that the fusion peptide has an anti-tumour effect derived from the melittin, and also is selective for cells over-expressing the integrins αvβ6 and/or αvβ3, its selectivity is enhanced from the RGD motif containing peptide.

In an aspect, in order to facilitate the bi-functional characteristics of the melittin fusion peptides, the melittin is directly fused or bound to the RGD motif containing peptide without a linker.

In an alternative embodiment, the melittin can be fused or bound to the RGD motif containing peptide via a linker. Linkers can in some embodiments assist with improving the specificity of delivery to a particular tumour site. Preferably, the linker is cleavable in vivo. Most preferably, the linker is cleaved at the tumour site.

### (d) Melittin Fusion Peptide Variants

In some embodiments, the melittin fusion peptide comprises one or more amino acid substitutions of the melittin fusion peptides discussed above. A skilled person will be able to determine suitable variants of the melittin fusion peptides which retain functionality, using well-known techniques.

In certain other embodiments, one skilled in the art can identify suitable areas of the peptide that can be modified without destroying activity by, for example, targeting regions not believed to be important for activity. For example, such area can be modified by conservative amino acid substitutions without destroying the biological activity or without adversely affecting the polypeptide structure.

Most preferably, the C-terminus of the melittin fusion peptides of this invention and their variants retain a positive charge. In some embodiments, the retention of the positive charge is advantageous for maintaining the biological activity of the melittin fusion peptide. Preferably, the melittin fusion peptides of the invention and their variants do not comprise a negatively charged C-terminus sequence.

In a particularly preferred form of the invention, a variant melittin fusion peptide retains the original positive K₂₁RKR₂₄ (SEQ ID NO: 4) sequence of the C-terminus of melittin of SEQ ID NO: 1. In a highly preferred form of the invention, melittin fusion peptide variants that retain SEQ ID NO: 4 sequence at the C-terminus exhibit the biological activities characteristic of melittin and melittin fusion peptides of SEQ ID NO: 3.

In another form of the invention, the melittin fusion peptide comprises an alternative C-terminus sequence that is positively charged. In an embodiment, the melittin fusion peptide comprises a C-terminal sequence of ((K₂₁KKRKV₂₆) present in the Simian Virus 40 (SV40) large T antigen (peptide SV40-melittin)) (SEQ ID NO:5), derived from the Simian Virus 40 (SV40) large T antigen (peptide SV40-melittin).

The melittin fusion peptides of this invention can be produced by routine and standard techniques known in the art. For example, the melittin fusion peptides can be synthesised through the use of peptide synthesizers well known in the art, or through standard recombinant DNA technology known in the art.

Peptides encompassed by the present invention include peptide variants based on a common scaffold, wherein each peptide variant comprises an amino acid sequence with an amino acid sequence identity compared to the primary peptide sequence selected from the group consisting of: 99% or greater identity; 98% or greater identity; 97% or greater identity; 96% or greater identity; 95% or greater identity; 90% or greater identity; 85% or greater identity; 80% or greater identity; 75% or greater identity; and 70% or greater identity.

Preferably, any changes to the primary peptide sequence to create a variant peptide includes amino acid deletions, amino acid additions and/or amino acid substitutions.

Amino acid substitutions are preferably conservative amino acid substitutions known to those skilled in the art. For example, the person skilled in the art may perform an amino acid substitution by selecting an amino acid from within the same class of amino acid that is shared with the specific amino acid that is identified for substitution. The amino acid classes, where the amino acids are sorted into six main classes on the basis of their structure and general chemical characteristics of their side chains (R groups), are:
Class 1—Glycine, Alanine, Valine, Leucine, Isoleucine;
Class 2—Serine, Cysteine, Selenocysteine, Threonine, Methionine;
Class 3—Proline;
Class 4—Phenylalanine, Tyrosine, Tryptophan;
Class 5—Histidine, Lysine, Arginine;
Class 6—Aspartate, Glutamate, Asparagine, Glutamine

Substitutions within these classes are often known to be conservative.

As an alternative approach, the person skilled in the art may select amino acid substitutions based on the table below. These substitutions are also known to be conservative.

TABLE 1

| Amino acids | Examples of conservative substitutions |
|---|---|
| Ala (A) | Val, Leu, Ile |
| Arg (R) | Lys, Gln, Asn |
| Asn (N) | Gln |
| Asp (D) | Glu |
| Cys (C) | Ser, Ala |
| Gln (Q) | Asn |
| Glu (E) | Asp |
| Gly (G) | Pro, Ala |
| His (H) | Asn, Gln, Lys, Arg |
| Ile (I) | Leu, Val, Met, Ala, Phe, Norleucine |
| Leu (L) | Ile, Val, Met, Ala, Phe, Norleucine |
| Lys (K) | Arg, Gln, Asn |
| Met (M) | Leu, Ile, Phe |
| Phe (F) | Leu, Val, Ile, Ala, Tyr |
| Pro (P) | Ala, Gly |
| Ser (S) | Thr, Ala, Cys |
| Trp (W) | Phe, Tyr |
| Thr (T) | Ser |
| Tyr (Y) | Trp, Phe, Thr, Ser |
| Val (V) | Ile Met, Leu, Phe, Ala, Norleucine |

The term “% identity”, as used here, may for example be calculated as follows. The query sequence is aligned to the target sequence using the CLUSTAL W algorithm (Thompson et al, Nucleic Acids Research, 22:4673-4680 (1994)). A comparison is made over the window corresponding to one of the aligned sequences, for example the shortest. The window may in some instances be defined by the target sequence. In other instances, the window may be defined by the query sequence. The amino acid residues at each position are compared, and the percentage of positions in the query sequence that have identical correspondences in the target sequence is reported as % identity.

### C. Pharmaceutical Compositions

In an aspect of the invention, the invention provides a pharmaceutical composition comprising a melittin fusion peptide of the invention. In a preferred embodiment, the melittin fusion peptide is of SEQ ID NO: 3 or a variant thereof. Preferably, the pharmaceutical composition additionally comprises pharmaceutically acceptable diluent, carrier, solubilizer, emulsifier, preservative and/or adjuvant.

The optimal pharmaceutical composition can be determined by a person skilled in the art depending upon, for example, the intended route of administration, delivery format and desired dosage. Such compositions may influence the physical state, stability, rate of in vivo release and rate of in vivo clearance of the melittin fusion peptides of the invention.

In an aspect, the primary vehicle or carrier in a pharmaceutical composition can be either aqueous or non-aqueous in nature. For example, in certain embodiments, a suitable vehicle or carrier can be water for injection, physiological saline solution or artificial cerebrospinal fluid, possibly supplemented with other materials common in compositions for parenteral administration. In some embodiments, the saline comprises isotonic phosphate-buffered saline. In some embodiments, neutral buffered saline or saline mixed with serum albumin are further exemplary vehicles.

The pharmaceutical composition components are present in concentrations that are acceptable to the site of administration. In certain embodiments, buffers are used to maintain the composition at physiological pH or at a slightly lower pH, typically within a pH range of from about 5 to about 8.

The pharmaceutical composition of this invention may be administered by intravenous, intraarterial, or intramuscular injection of a liquid preparation. Suitable liquid compositions include solutions, suspensions, dispersions, emulsions, oils and the like.

In embodiments when parenteral administration is contemplated, a therapeutic composition can be in the form of a pyrogen-free, parenterally acceptable aqueous solution comprising a desired melittin fusion peptide, with or without additional therapeutic agents, in a pharmaceutically acceptable vehicle. In certain embodiments, a vehicle for parenteral injection is sterile distilled water in which a melittin fusion peptide, with or without at least one additional therapeutic agent, is formulated as a sterile, isotonic solution, properly preserved. In certain embodiments, the preparation can involve the formulation of the desired melittin fusion peptide with an agent, such as injectable microspheres, bio-erodible particles, polymeric compounds (such as polylactic acid or polyglycolic acid), beads, nanoparticles, or liposomes, that can provide for the controlled or sustained release of the product which can then be delivered via a depot injection. In certain embodiments, hyaluronic acid can also be used, and can have the effect of promoting sustained duration in the circulation. In certain embodiments, implantable drug delivery devices can be used to introduce the desired molecule.

In certain embodiments, once the pharmaceutical composition has been formulated, it can be stored in sterile vials as a solution, suspension, gel, emulsion, solid, or as a dehydrated or lyophilized powder. In certain embodiments, such formulations can be stored either in a ready-to-use form or in a form (e.g., lyophilized) that is reconstituted prior to administration.

In some embodiments, the pharmaceutical composition comprises preservatives, including water soluble preservatives. Water soluble preservatives that may be employed include, but are not limited to, benzalkonium chloride, chlorobutanol, thimerosal, sodium bisulfate, phenylmercuric acetate, phenylmercuric nitrate, ethyl alcohol, methylparaben, polyvinyl alcohol, benzyl alcohol and phenylethyl alcohol.

The pharmaceutical composition herein may also contain additional therapeutic agents, preferably with a complementary activity that does not adversely affect the functioning of the melittin fusion peptide. Preferably, the pharmaceutical composition further comprises a chemotherapeutic agent or other anticancer therapy. Such molecules are suitably present in combination in amounts that are effective for the purpose intended.

In an embodiment, the pharmaceutical composition further comprises one or more chemotherapeutic agents. Preferably, the chemotherapeutic agent is docetaxel, trastuzumab, pertuzumab and/or cisplatin: see for example, Swain, S. M. et al. Pertuzumab, Trastuzumab, and Docetaxel in HER2-Positive Metastatic Breast Cancer. N. Engl. J. Med. 372, 724-734 (2015), which is hereby incorporated by reference in it's totality. In another embodiment, the pharmaceutical composition comprises an anticancer therapy. Preferably, the anticancer therapy is omomyc or a Cas9-gRNA complex or variant thereof.

### D. Dosage

The melittin fusion peptides, melittin and chemotherapeutic agents, and other anticancer therapeutics (and composition comprising these agents) herein can be administered for prophylactic and/or therapeutic treatments. In therapeutic applications, the compositions are administered to a patient already suffering from a disease or condition, in an amount sufficient to cure or at least partially arrest the symptoms or progression of the disease or condition. Amounts effective for this use will depend on the severity and course of the disease or condition, previous therapy, the patient's health status, weight, and response to the drugs, and the judgment of the treating physician. It is considered well within the skill of the art for one to determine such therapeutically effective amounts by routine experimentation (including, but not limited to, a dose escalation clinical trial).

In prophylactic applications, the melittin fusion peptides, melittin, chemotherapeutic agents and other anticancer therapeutics (and compositions comprising these agents) described herein are administered to a patient susceptible to or otherwise at risk of a particular disease, disorder, or condition. For example, in some embodiments, there is an increase in the expression of αvβ3, αvβ5, αvβ6, αvβ8, α5β1, and/or αIIbβ3 integrins during tumour formation which can be targeted by the melittin fusion peptides of the invention. Such an amount is defined to be a “prophylactically effective amount or dose”. In this use, the precise amounts also depend on the patient's state of health, weight, and the like. It is considered well within the skill of the art for one to determine such prophylactically effective amounts by routine experimentation (e.g., a dose escalation clinical trial). When used in a patient, effective amounts for this use will depend on the severity and course of the disease, disorder or condition, previous therapy, the patient's health status and response to the drugs, and the judgment of the treating physician.

In certain embodiments, a typical dosage can range from about 1 μg/Kg to up to about 10 mg/Kg or more, depending on the factors mentioned above. In certain embodiments, the dosage can range from 1 μg/Kg up to about 10 mg/Kg; or 10 μg/Kg up to about 10 mg/Kg; or 100 μg/Kg up to about 10 mg/Kg. Preferably the human does is in the range of from 0.5 mg/Kg or 500 μg/kg.

Preferably, the melittin or melittin fusion peptide is administered at a dosage of about 10 μg/Kg to 10 mg/Kg, more preferably at a dosage between 100 μg/Kg to 1 mg/Kg. In some embodiments, it is administered at a dosage of 500 μg/Kg.

Preferably, the chemotherapeutic agent is administered at a dosage of about 10-200 mg/m² IV every three weeks, more preferably at a dosage between 60-100 mg/m² IV every three weeks. In some embodiments, it is administered at a dosage of 75 mg/m² IV every three weeks. Preferably the chemotherapeutic agent is docetaxel or cisplatin.

In another embodiment, the chemotherapeutic agent is carboplatin. Preferably, the carboplatin is administered to a subject suffering from triple negative breast cancer in six cycles of carboplatin with target AUC 6, every 3 weeks until disease progression, and the melittin fusion peptide is administered at a dosage of 0.5 mg/Kg to 100 mg/Kg.

In some aspects, the anticancer therapy additionally comprises treatment with pertuzumab or trastuzumab. Preferably, the pertuzumab is administered at a dose of 840 mg in the first cycle, and then at 420 mg every three weeks. Preferably, trastuzumab is administered at a dose of 8 mg/Kg in the first cycle and at 6 mg/Kg every 3 weeks.

In other aspects, the anticancer therapy is omomyc. Preferably, omomyc is administered at a dosage of 0.5 mg/Kg to 10 mg/Kg. Most preferably, omomyc is administered at a dosage of 2.6 mg/Kg, as set out in Wang et. al. Oncogene (2019) 38:140-150 which is incorporated by reference. In another aspect, the anticancer therapy is a Cas9-gRNA complex or variant thereof. Preferably, the Cas9-gRNA complex or variant thereof is administered at a dose of 250 pM-1000 pM. Most preferably, the Cas9-gRNA complex or variant thereof is administered at a dose of 250 pM.

The melittin, melittin fusion peptide or pharmaceutical composition comprising the melittin fusion peptide can be administered to the subject in a range of treatment regimens. For example, the melittin, melittin fusion peptide or pharmaceutical composition can be administered hourly, three times daily, twice daily, once daily, once every two days, once every three days, once weekly, once every two weeks, once monthly, once every two months, once every six months, and once yearly. The appropriate regimen can be determined by the person skilled in the art based on the nature of the condition to be treated. For example, the melittin, melittin fusion peptide or pharmaceutical composition comprising the melittin fusion peptide can be administered every two days.

### E. Method of Treatment

In another aspect, the invention provides a method of treating cancer in a subject comprising administering to said subject a therapeutically effective amount of a melittin fusion peptide of the invention.

Preferably the cancer to be treated is any tumour cell sub-type wherein the integrins αvβ6 and/or αvβ3 are overexpressed on the plasma membrane of the tumour cell and/or associated vasculature. Preferably, the cancer is selected from the group of HER2-enriched breast cancer, triple negative breast cancer or ovarian cancer.

Preferably, the method of treating cancer additionally comprises the step of administering to said subject an anticancer therapy.

Anticancer therapies include but are not limited to natural killer cell-based immunotherapy cells (NK-92 MI), HER2-targeted agents including monoclonal antibodies, trastuzumab-emtansine, other antibody-drug conjugates, ribonucleoproteins such as Cas9-gRNA and variants, or polypeptides such as omomyc where the membrane disrupting properties of melittin could enhance the internalization kinetics of the cytotoxic payload. It is to be understood that anticancer therapies for use in the present invention also include novel compounds or treatments developed in the future.

Preferably, the anticancer therapy is a chemotherapeutic agent and is selected from docetaxel, cisplatin, pertuzumab, and/or trastuzumab.

In another preferred embodiment, the anticancer therapy is omomyc. Omomyc is a dominant-negative MYC mutant comprising the b-HLH-LZ domain of Myc, with four amino acid substitutions in the leucine zipper of Myc (Soucek, L. et al., 1998, Oncogene 17, 2463-2472; Soucek, L, et al. (2002), Cancer Res 62:3507-3510). These substitutions (E61T, E681, R74Q, and R75N) confer altered dimerization specificity to Myc, which retains the ability to heterodimerize with its natural partner Max and to form homodimers with wild type c-, N- and L-Myc. Omomyc negates the ability of Myc to bind its DNA recognition binding site, the E box, and is able to prevent Myc-dependent gene transactivation functions both in vitro and in vivo. Omomyc also strongly enhances Myc-induced apoptosis dependent on Myc expression level to strengthen Myc transrepression activity. Therefore, while retaining Miz-1 dependent binding to promoters and transrepression, omomyc prevents Myc binding to promoter E-boxes and transactivation of target genes. In the presence of omomyc, the activity of the Myc interactome changes from pro-oncogenic to tumor-suppressive.

In another preferred embodiment, the anticancer therapy is a Cas9-gRNA complex or variant such as a dCas9-gRNA complex. These complexes consist of two parts: a guide RNA (gRNA) and a Cas9 binding endonuclease. The guide RNA is a short synthetic RNA consisting of a scaffold sequence necessary for binding to Cas9, and a specific targeting sequence (target site) that is specific for the target location. Cas9-gRNA complexes can be used to edit genes involved in tumour proliferation. Variants of Cas9-gRNA complexes such as catalytically inactive dCas9-gRNA can also be used for the purpose of inducing epigenetic modifications, in which dCas9 or variants are linked to domains that modifies chromatin.

In another aspect, the invention provides a melittin fusion peptide of the invention for use in a method of treating cancer in a subject in need thereof. Preferably the cancer to be treated is any tumour cell sub-type wherein the integrins αvβ6 and/or αvβ3 are overexpressed on the plasma membrane of the tumour cell and/or associated vasculature. Preferably, the cancer is selected from the group of HER2-enriched breast cancer, triple negative breast cancer or ovarian cancer.

In another aspect, the invention provides the use of a melittin fusion peptide of the invention in the manufacture of a medicament for treating cancer in a subject in need thereof. Preferably the cancer to be treated is any tumour cell sub-type wherein the integrins αvβ6 and/or αvβ3 are overexpressed on the plasma membrane of the tumour cell and/or associated vasculature. Preferably, the cancer is selected from the group of HER2-enriched breast cancer, triple negative breast cancer or ovarian cancer.

In a further aspect, the invention provides a method of treating triple negative breast cancer, HER2 enriched breast cancer or ovarian cancer in a subject comprising administering to said subject a therapeutically effective amount of a combination therapy of a melittin fusion peptide and docetaxel, cisplatin, pertuzumab, and/or trastuzumab.

The invention also provides a method of reducing tumour cell viability in a subject, said method comprising the step of administration of a therapeutically effective amount of a melittin fusion peptide of the invention. Preferably the melittin fusion peptide comprises SEQ ID NO: 3 or a variant thereof. Preferably, the method further comprises the step of administering to said subject a chemotherapeutic agent or other anticancer therapy. Preferably, the chemotherapeutic agent is selected from docetaxel, cisplatin, pertuzumab, omomyc and/or trastuzumab. In another preferred embodiment, the anticancer therapy is selected from omomyc or Cas9-gRNA complexes or a variant thereof.

Further, the invention provides a method of inhibiting PD-L1 expression in a subject suffering from cancer comprising the step of administering melittin or a melittin fusion peptide in a therapeutically effective amount to the subject. Preferably, the method additionally comprises the step of administering a chemotherapeutic agent. More preferably, the chemotherapeutic agent is docetaxel, cisplatin, pertuzumab, omomyc and/or trastuzumab. Preferably, the subject is suffering from triple negative breast cancer, HER2-enriched breast cancer or ovarian cancer.

Immune checkpoint protein PD-L1 binds to the immune checkpoint protein PD-1, and can inhibit the activation and immune recognition of T-cells that would otherwise be involved in the natural immune response against tumour cells. The inhibition of PD-L1 results in the activation of T cells. It has also been shown that in certain cancers, the administration of certain chemotherapeutic agents can increase the expression of PD-L1, resulting in reduced immune response to the tumour cell, and reduced overall efficacy of the treatment.

In this aspect of the invention, the administration of a therapeutically effective amount of melittin or melittin fusion peptide to a subject suffering from cancer reduces the expression of PD-L1 in tumour cells. This can result in improved anti-tumoral immune responses by the subject.

Preferably the cancer to be treated is any tumour cell sub-type wherein the integrins αvβ6 and/or αvβ3 are overexpressed on the plasma membrane of the tumour cell and/or associated vasculature. Preferably, the cancer is selected from the group of HER2-enriched breast cancer, triple negative breast cancer or ovarian cancer.

In another aspect, the invention provides a melittin fusion peptide of the invention for use in a inhibiting PD-L1 expression in a subject suffering from cancer. Preferably the cancer is any tumour cell sub-type wherein the integrins αvβ6 and/or αvβ3 are overexpressed on the plasma membrane of the tumour cell and/or associated vasculature. Preferably, the cancer is selected from the group of HER2-enriched breast cancer, triple negative breast cancer or ovarian cancer.

In a preferred embodiment of this aspect of the invention, the method additionally comprises the administration of an anticancer therapy.

In another aspect, the invention provides the use of a melittin fusion peptide of the invention in the manufacture of a medicament for inhibiting PD-L1 expression in a subject suffering from cancer. Preferably the cancer is any tumour cell sub-type wherein the integrins αvβ6 and/or αvβ3 are overexpressed on the plasma membrane of the tumour cell and/or associated vasculature. Preferably, the cancer is selected from the group of HER2-enriched breast cancer, triple negative breast cancer or ovarian cancer.

In a further aspect, the invention provides a method of facilitating the cell penetration of an anticancer therapy in a subject suffering from cancer, comprising the step of administering a therapeutically effective amount of a combination therapy of a melittin fusion peptide and an anticancer therapy to the subject.

Preferably, the anticancer therapy is omomyc or a Cas9-gRNA complex or variant thereof.

Preferably the cancer to be treated is any cancer cell sub-type wherein the integrins αvβ6 and/or αvβ3 are overexpressed. Most preferably, the cancer is selected from the group of HER2-enriched, triple negative breast cancer or ovarian cancer.

### F. Combination Therapy

Another aspect of the present invention provides a method for treating cancer in a subject comprising the step of administering to the said subject a therapeutically effective amount of a melittin fusion peptide of the invention (or pharmaceutical composition comprising a melittin fusion peptide) and additionally the step of administering to said subject an anticancer therapy. The melittin fusion peptide can be administered before, during or after treatment with the anticancer therapy in order to treat the subject in need thereof.

In another aspect of the invention, there is provided a method for reducing the expression of PD-L1 in a subject suffering from cancer, and additionally the step of administering to said subject an anticancer therapy.

Preferably, the anticancer therapy is a chemotherapeutic agent. Preferably, the chemotherapeutic agent is an agent that is used in the standard of care for the type of cancer suffered by the subject. Most preferably, the chemotherapeutic agent is selected from docetaxel, trastuzumab, pertuzumab and/or cisplatin, used to treat a patient with triple negative breast cancer, HER2 enriched breast cancer or ovarian cancer. Alternatively, the chemotherapeutic agent is carboplatin.

In another embodiment, the anticancer therapy is a polypeptide. Preferably, the polypeptide is omomyc.

In a further embodiment, the anticancer therapy is a Cas9-gRNA complex or a variant thereof.

In some embodiments, the melittin fusion peptide is administered together with docetaxel. Preferably, docetaxel is administered to a subject suffering from triple negative breast cancer or HER2 enriched breast cancer at a dosage of 60 to 100 mg/m² IV every three weeks until disease progression is arrested, and the melittin fusion peptide is administered at a dosage of 0.5 to 100 mg/Kg, in order to treat triple negative breast cancer or HER2 enriched breast cancer. Preferably, docetaxel is administered at a dose of 75 mg/m² on day 2, and then at 75 mg/m² every 3 weeks, with dose adjustments allowed up to 100 mg/m², or a 25% dose reduction to improve tolerability. Preferably, docetaxel is administered together with one or more additional chemotherapeutic agents. Preferably, the additional chemotherapeutic agent is trastuzumab and/or pertuzumab.

Preferably, trastuzumab is administered in combination with pertuzumab or another like antibody in accordance with the methodology set out in Swain, S. M. et al. Pertuzumab, Trastuzumab, and Docetaxel in HER2-Positive Metastatic Breast Cancer. N. Engl. J. Med. 372, 724-734 (2015), to a subject suffering from HER2 positive metastatic breast cancer.

Most preferably, the trastuzumab is administered at a dosage of 8 mg/Kg on day 1, then at 6 mg/Kg every three weeks until disease progression is arrested, and the melittin fusion peptide is administered at a dosage of 0.5 to 100 mg/Kg, in order to treat the breast cancer.

In some aspects, the anticancer therapy additionally comprises treatment with pertuzumab or trastuzumab. Preferably, the pertuzumab is administered at a dose of 840 mg on day 1, and then at 420 mg every three weeks. Preferably, trastuzumab is administered at a dose of 8 mg/Kg on day 1, and at 6 mg/Kg every 3 weeks.

In some aspects, the melittin fusion peptide of the invention is administered together with cisplatin. Preferably, cisplatin or combinations of cisplatin with other agents is (are) administered to a subject suffering from triple negative breast cancer, HER2 enriched breast cancer or ovarian cancer at a cisplatin dosage of 60 to 100 mg/m² IV every three weeks until disease progression is arrested, and the melittin fusion peptide is preferably administered at a dosage of 0.5 to 100 mg/Kg.

In another aspect, the melittin fusion peptide is administered together with carboplatin. Preferably, the carboplatin is administered to a subject suffering from triple negative breast cancer, HER2 enriched breast cancer or ovarian cancer. Carboplatin is administered over six cycles with target AUC 6, every 3 weeks or until disease progression has been arrested. The melittin fusion peptide is preferably administered at a dosage of 0.5 to 100 mg/Kg.

In another embodiment, the melittin fusion peptide is administered together with omomyc. Preferably, the omomyc is administered at a dosage of about 10 μg/Kg to 10 mg/Kg, more preferably at a dosage between 100 μg/Kg to 10 mg/Kg. In some embodiments, it is administered at a dosage of 2.6 mg/Kg. Preferably, omomyc is administered at a dosage of 0.5 mg/Kg to 10 mg/Kg. Most preferably, omomyc is administered at a dosage of 2.6 mg/Kg.

In another embodiment, the melittin fusion peptide is administered together with a Cas9-gRNA complex or a variant thereof. Preferably, the a Cas9-gRNA complex or a variant thereof is administered at a dosage of about 10 μg/Kg to 10 mg/Kg, more preferably at a dosage between 100 μg/Kg to 1 mg/Kg. Preferably, the Cas9-gRNA complex or variant thereof is administered at a dose of 250 pM-1000 pM. Most preferably, the Cas9-gRNA complex or variant thereof is administered at a dose of 250 pM.

In a further aspect, the invention provides a method of facilitating the cell penetration of an anticancer therapy in a subject suffering from cancer, comprising the step of administering a therapeutically effective amount of a combination therapy of a melittin fusion peptide and an anticancer therapy to the subject.

The anticancer therapy is any compound, composition or treatment that prevents or delays the growth and/or metastasis of cancer cells, and can include small molecules, peptides, polypeptides and protein-nucleic acid complexes, including those that are larger than 100 kDa.

Without being bound by theory, the melittin fusion peptide can generate transmembrane pores with an internal diameter of approximately 4.4 nm. These pores may facilitate the cell penetration of other therapeutics, including anticancer therapies (including chemotherapeutic agents). This can be of particular benefit in respect of anticancer therapies that otherwise do not easily penetrate into tumour cells, for example, due to their size.

In some aspects there is a synergistic or beneficial action between the melittin fusion peptide and the anticancer therapy. Preferably, the anticancer therapy is a chemotherapeutic agent, specifically docetaxel, trastuzumab, pertuzumab and/or cisplatin. In another preferred embodiment, the anticancer therapy is omomyc or Cas9-gRNA or a variant thereof.

In a particularly preferred embodiment, the melittin fusion peptide of the above methods comprises SEQ ID NO: 3 or a variant thereof.

In a further aspect, the present invention provides a method for treating cancer in a subject comprising the step of administering to the said subject a therapeutically effective amount of melittin (or pharmaceutical composition comprising melittin) and additionally the step of administering to said subject an anticancer therapy. The melittin can be administered before, during or after treatment with the anticancer therapy in order to treat the subject in need thereof.

Preferably, the therapeutically effective amount of melittin is administered together with a therapeutically effective amount of cisplatin, pertuzumab, trastuzumab, and/or docetaxel to treat a patient suffering from cancer. More preferably, the therapeutically effective amount of melittin is administered together with the therapeutically effective amount of cisplatin, pertuzumab, trastuzumab, and/or or docetaxel to treat a patient suffering from triple negative breast cancer, HER2 enriched breast cancer or ovarian cancer.

In another embodiment, the therapeutically effective amount of melittin is administered together with a therapeutically effective amount of omomyc, or a Cas9-gRNA complex or variant thereof. More preferably, the therapeutically effective amount of melittin is administered together with the therapeutically effective amount of omomyc, or a Cas9-gRNA complex or variant thereof to treat a patient suffering from triple negative breast cancer, HER2 enriched breast cancer or ovarian cancer.

In a further aspect, the invention provides a method of facilitating the cell penetration of an anticancer therapy in a subject suffering from cancer, comprising the step of administering a therapeutically effective amount of a combination therapy of melittin and an anticancer therapy to the subject.

The anticancer therapy is any compound, composition or treatment that prevents or delays the growth and/or metastasis of cancer cells, and can include small molecules, peptides, polypeptides and protein-nucleic acid complexes more, including those that are larger than 100 kDa.

Without being bound by theory, melittin can generate transmembrane pores with an internal diameter of approximately 4.4 nm. These pores may facilitate the cell penetration of other therapeutics, including anticancer therapies (including chemotherapeutic agents). This can be of particular benefit in respect of anticancer therapies that otherwise do not easily penetrate into tumour cells, for example, due to their size.

In some aspects there is a synergistic or beneficial action between the melittin and the anticancer therapy. Preferably, the anticancer therapy is a chemotherapeutic agent, specifically docetaxel, trastuzumab, pertuzumab and/or cisplatin. In another preferred embodiment, the anticancer therapy is omomyc or Cas9-gRNA or a variant thereof.

In another aspect, the invention provides a method of inhibiting PD-L1 expression in a subject suffering from cancer comprising the step of administering melittin or a melittin fusion peptide in a therapeutically effective amount to the subject, and the step of administering a chemotherapeutic agent. More preferably, the chemotherapeutic agent is docetaxel, trastuzumab, pertuzumab, and/or cisplatin.

The administration of a therapeutically effective amount of melittin or melittin fusion peptide to a subject suffering from cancer reduces the expression of PD-L1 in tumour cells, which can otherwise be upregulated by the administration of some chemotherapeutic agents, including trastuzumab. This can result in improved anti-tumoral immune responses by the subject. In the combination therapy, the administration of melittin or a melittin fusion peptide in a therapeutically effective amount can result in improved anti-tumoral immune responses by the subject, and reduce any upregulation of PD-L1 expression by the chemotherapeutic agent in the subject. Preferably, the chemotherapeutic agent is docetaxel, trastuzumab, pertuzumab, and/or cisplatin and the subject is suffering from triple negative breast cancer, HER2-enriched breast cancer or ovarian cancer.

### G. Kits

In an embodiment, the invention provides a kit for treating cancer comprising a melittin fusion peptide together with instructions as to how to use that fusion peptide in accordance with this invention.

In one preferred embodiment, the melittin fusion peptide is of SEQ ID NO: 3 or a variant thereof. Preferably, the kit can additionally comprise a chemotherapeutic agent. More preferably, the chemotherapeutic agent is docetaxel, trastuzumab, pertuzumab, and/or cisplatin. In another preferred embodiment, the kit can additionally comprise another anticancer therapy. More preferably, the anticancer therapy is omomyc or Cas9-gRNA. Individual components of the kit can be packaged in separate containers and, associated with such containers, can be a notice in the form prescribed by a governmental agency regulating the manufacture, use or sale of pharmaceuticals or biological products, which notice reflects approval by the agency of manufacture, use or sale for human administration.

When the components of the kit are provided in one or more liquid solutions, the liquid solution can be an aqueous solution, for example a sterile aqueous solution. For in vivo use, the expression construct may be formulated into a pharmaceutically acceptable syringeable composition. In this case the container means may itself be an inhalant, syringe, pipette, eye dropper, or other such like apparatus, from which the formulation may be applied to an affected area of the animal, such as the breast, injected into an animal, or even applied to and mixed with the other components of the kit.

The components of the kit may also be provided in dried or lyophilized forms. When reagents or components are provided as a dried form, reconstitution generally is by the addition of a suitable solvent. It is envisioned that the solvent also may be provided in another container means. Irrespective of the number or type of containers, the kits of the invention also may comprise, or be packaged with, an instrument for assisting with the injection/administration or placement of the ultimate complex composition within the body of an animal or patient. Such an instrument may be an inhalant, syringe, pipette, forceps, measured spoon, eye dropper or any such medically approved delivery vehicle.

### H. Nucleic Acid Molecules

In an embodiment, the invention comprises a nucleic acid molecule comprising the melittin fusion peptides of this invention. In a preferred embodiment, the nucleic acid molecule comprises SEQ ID NO: 7 or 8, which are nucleic acid sequences encoding the melittin fusion peptide of SEQ ID NO: 3:

Homo sapiens:
SEQ ID NO: 7
CACGGCAGGGGCGACCTGGGCAGGCTGAAGAAGGGCATCGGCGCCGTGCT
GAAGGTGCTGACCACCGGCCTGCCCGCCCTGATCAGCTGGATCAAGAGGA
AGAGGCAGCAG
E. coli:
SEQ ID NO: 8
CATGGCCGCGGCGATCTGGGCCGCCTGAAAAAAGGCATTGGCGCGGTGCT
GAAAGTGCTGACCACCGGCCTGCCGGCGCTGATTAGCTGGATTAAACGCA
AACGCCAGCAG

One of skill in the art will appreciate that the above discussion can be used for identifying, evaluating, and/creating melittin fusion peptides and also for nucleic acid sequences that can encode for those peptides. Thus, nucleic acid sequences encoding for those peptides are contemplated.

In an embodiment, the invention comprises a vector comprising a nucleic acid molecule as described herein.

In an embodiment, the invention comprises a host cell comprising a nucleic acid molecule as described herein.

### I. Selective Binding Agents

As used herein, the term “selective binding agent” refers to a molecule that has specificity for peptides of the invention described herein. Suitable selective binding agents include, but are not limited to, antibodies and derivatives thereof, polypeptides and small molecules. Suitable selective binding agents may be prepared using methods known in the art. An exemplary selective binding agent is capable of binding a portion of a peptide of the invention. Such binding agents can be utilised to determine the presence of peptides of the invention in tissue or individual cells and determine binding activity and/or localisation with other molecules.

Selective binding agents such as antibodies and antibody fragments that bind peptides of the invention include monospecific polyclonal, monoclonal (MAbs), recombinant, chimeric, humanized such as CDR-grafted, human, single chain, and/or bispecific, as well as fragments, variants or derivatives thereof. Antibody fragments include those portions of the antibody that bind to an epitope on the peptide. Examples of such fragments include Fab and F(ab′) fragments generated by enzymatic cleavage of full-length antibodies. Other binding fragments include those generated by recombinant DNA techniques, such as the expression of recombinant plasmids containing nucleic acid sequences encoding antibody variable regions.

The selective binding agents, including antibodies, are also useful for in vivo imaging of administered peptides of the invention. An antibody labelled with a detectable moiety may be administered to an animal, preferably into the bloodstream, and the presence and location of the labelled antibody in the host is assayed. The antibody may be labelled with any moiety that is detectable in an animal, whether by nuclear magnetic resonance, radiology, or other detection means known in the art.

The invention also relates to a kit comprising selective binding agents (such as antibodies) and other reagents useful for detecting the levels and localisation of the peptides described herein in biological samples. Such reagents may include, a detectable label, blocking serum, positive and negative control samples, and detection reagents.

In particular, antibodies may be used to detect peptides of the invention present in biological samples. Suitable samples are preferably from breast or ovarian tissue but may also include extracts of tissues such as brain, skin, heart, lung, colon, pancreas, testes, liver, muscle, prostate and bone tissues. Such antibodies of the invention may be bound to a solid support and/or packaged into kits in a suitable container along with suitable reagents, controls, instructions and the like.

In an embodiment, the invention comprises an isolated monoclonal antibody that specifically binds to melittin of SEQ ID NO: 1 and/or SEQ ID NO: 3.

### EXAMPLES

Further features of the present invention are more fully described in the following non-limiting Examples. This description is included solely for the purposes of exemplifying the present invention. It should not be understood as a restriction on the broad description of the invention as set out above.

### Example 1

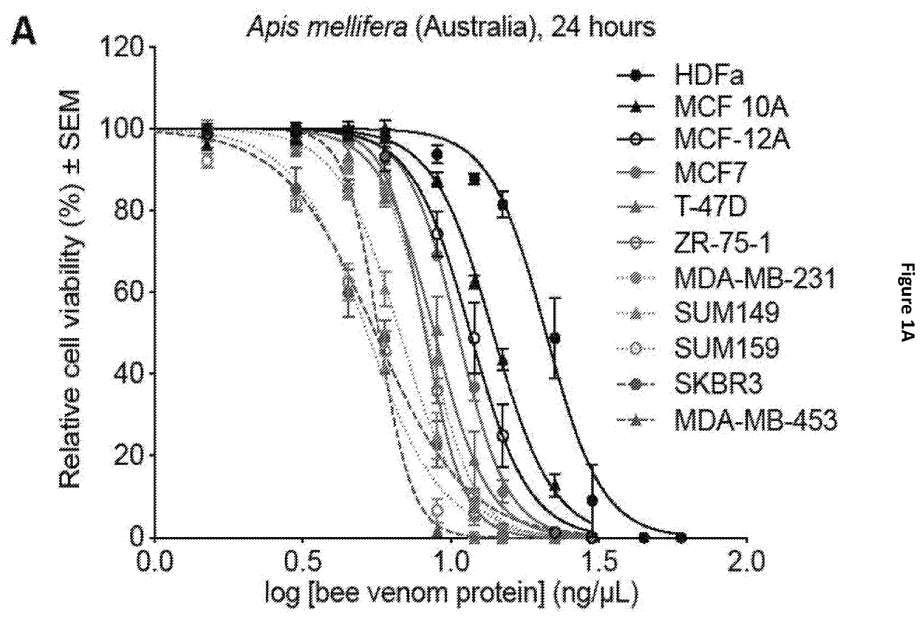

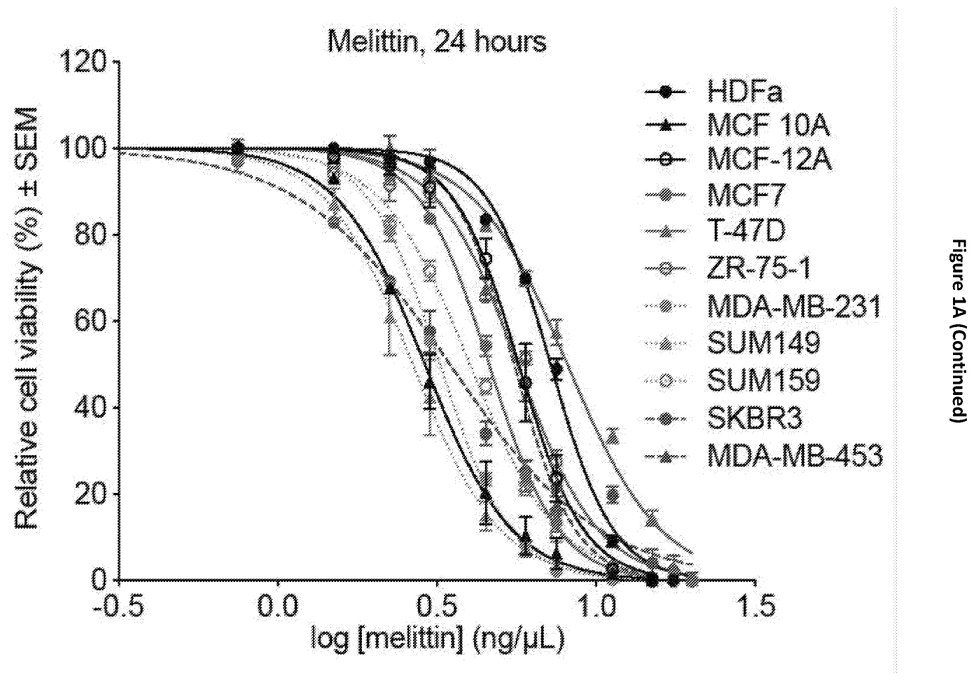

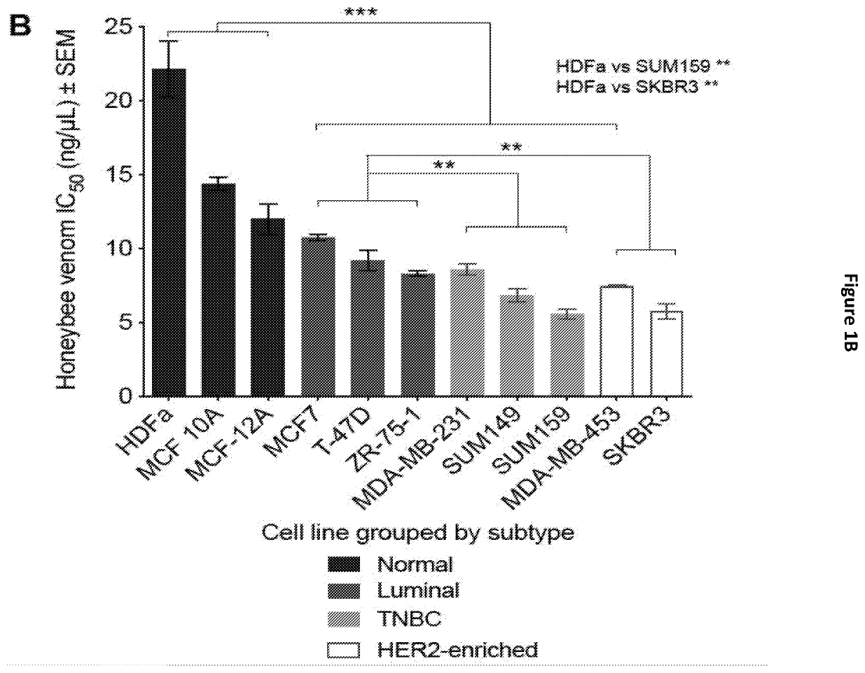

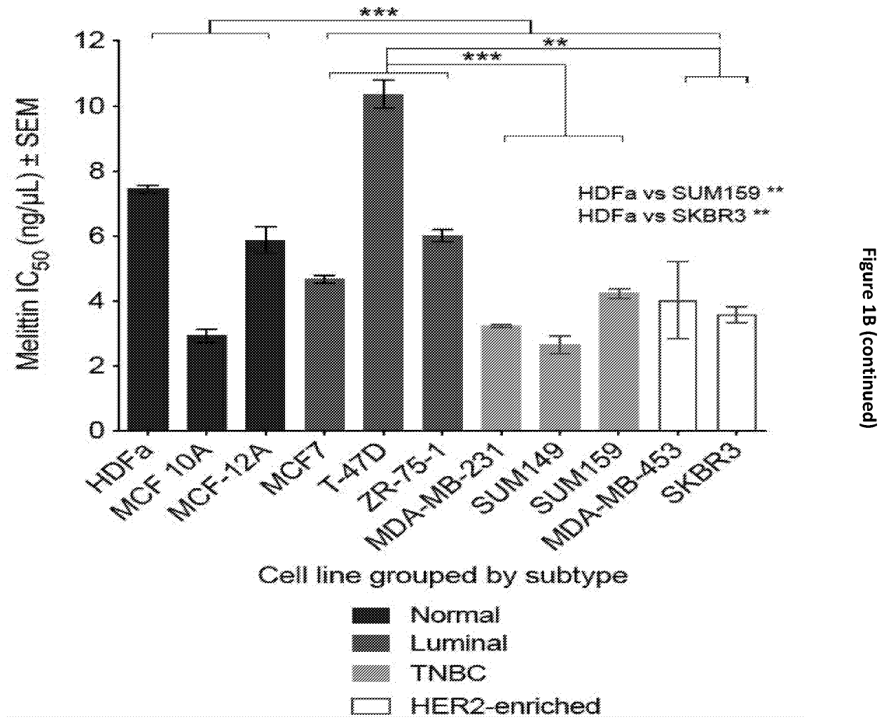

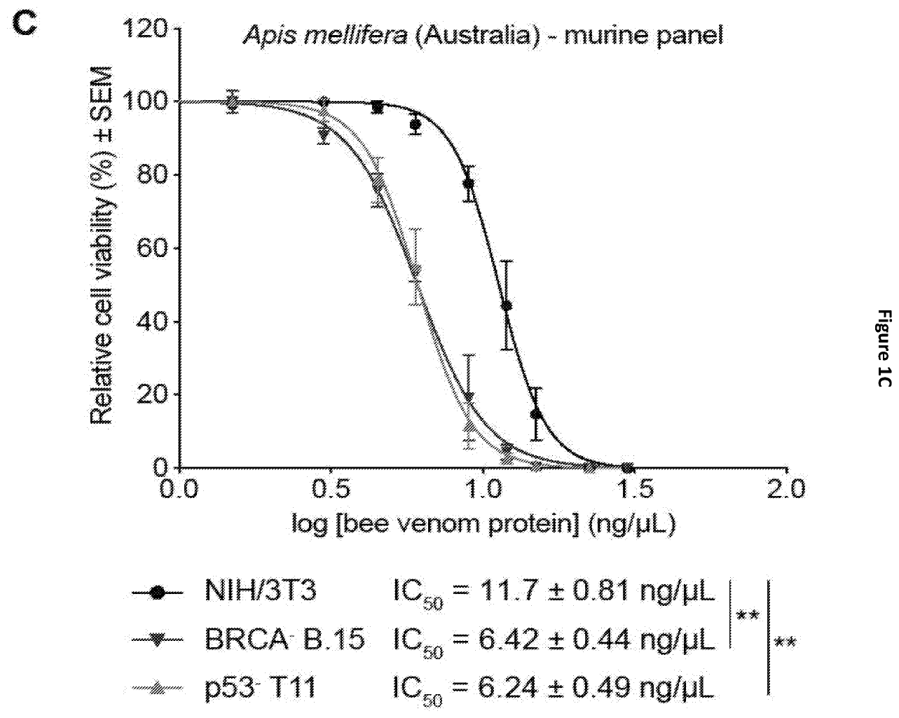

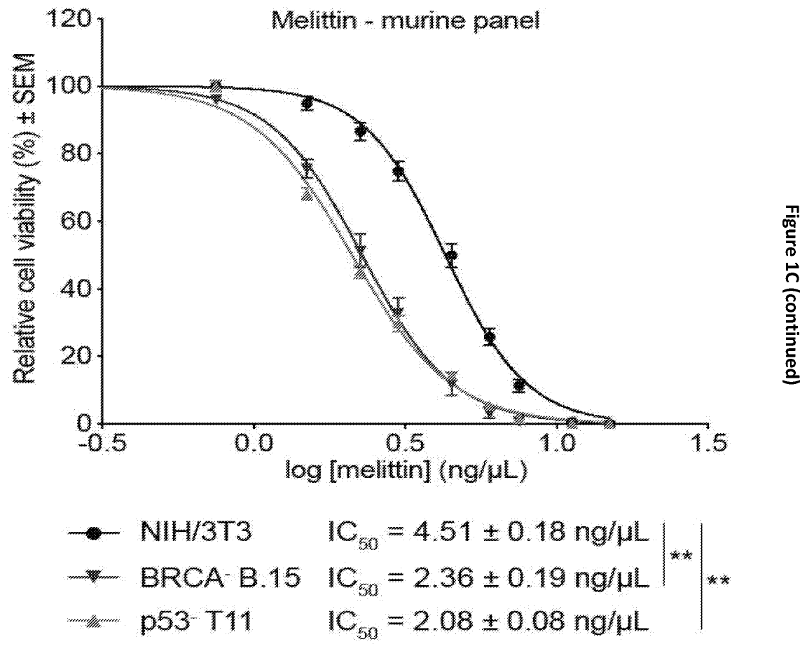

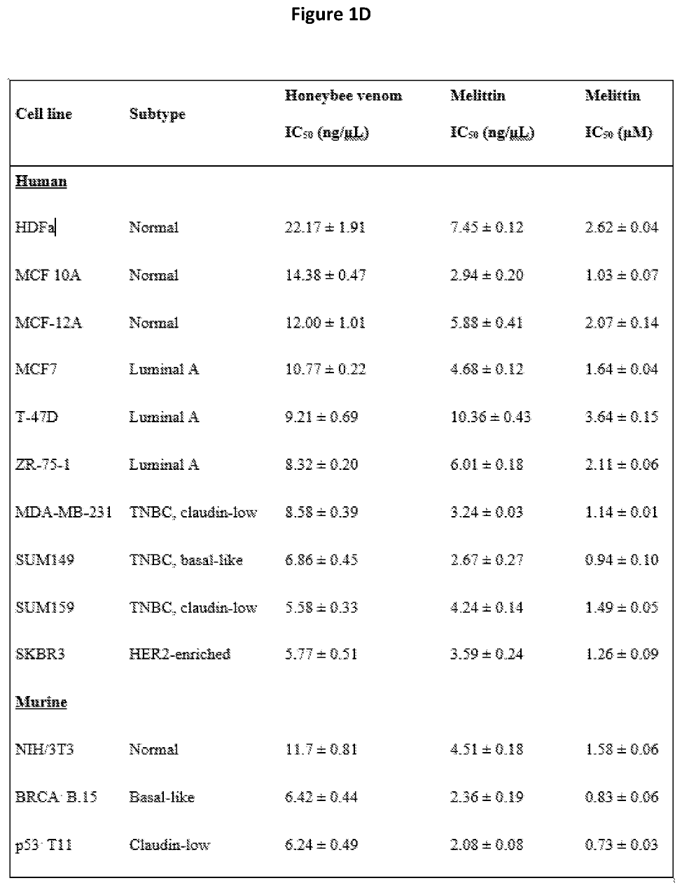

To assess anticancer efficacy and selectivity, both venom from European honeybees collected in Perth, Australia and melittin peptide were evaluated in dose-response assays in a panel of cell lines representative of the intrinsic breast cancer subtypes and in non-transformed cells. Honeybee venom showed high anticancer selectivity, with a significantly higher potency in TNBC (e.g. SUM159, SUM149) and in the HER2-enriched breast cancer cell lines (e.g. MDA-MB-453, SKBR3), followed by in luminal breast cancer cells (including MCF7 and T-47D), with the lowest impact on normal cells (primary dermal fibroblast cells HDFa, and mammary non-transformed MCF10A and MCF-12A cells) (FIG. 1A, left; Generalized Linear Model, p<0.001). A significant reduction in the half-maximal inhibitory concentration (IC₅₀) for both TNBC SUM159 (5.58 ng/μL) and HER2-enriched SKBR3 (5.77 ng/L) breast cancer cells was observed compared with the normal HDFa cell line (22.17 ng/μL; FIG. 1B, left; one-way ANOVA, p<0.01).

Similarly, melittin was significantly more potent against HER2-enriched breast cancer and TNBC compared to normal cells (FIG. 1A-B, right; Generalized Linear Model, p<0.001), with IC₅₀ values from 0.94-1.49 UM in human TNBC and HER2-enriched breast cancer cells, and 1.03-2.62 M in non-transformed cells. Cell viability assays of honeybee venom and melittin in murine breast cancer and normal cell lines confirmed enhanced selectivity for aggressive murine tumour cell lines, such as the p53-mutant claudin-low T11 and the BRCA-mutant B. 15 line (FIG. 1C). The IC₅₀ values for each of these dose-response assays are presented in FIG. 1D. The table shows the half-maximal inhibitory concentrations (IC50) of honeybee venom and melittin for the panel of breast normal and cancer cell lines tested in human and murine cell lines. Data are presented as mean±SEM in ng/μL or μM (to two decimal places). Experiments were performed in biological triplicate.

The venom of honeybees from different honeybee populations in Ireland and England reduced the viability of SUM159 and SKBR3 cells significantly more than that of non-transformed HDFa cells (FIG. 2A; one-way ANOVAs, p<0.001). The venom from the bumblebee Bombus terrestris from England was also tested, and samples from both workers and queens elicited minimal cell death in breast cancer cells compared to honeybee venom even at high concentrations of venom (FIG. 2B).

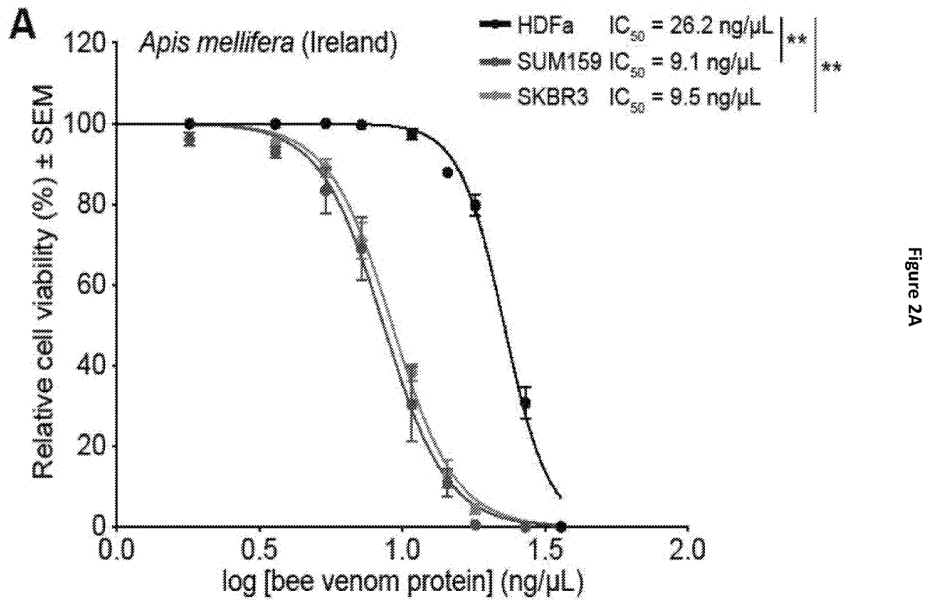

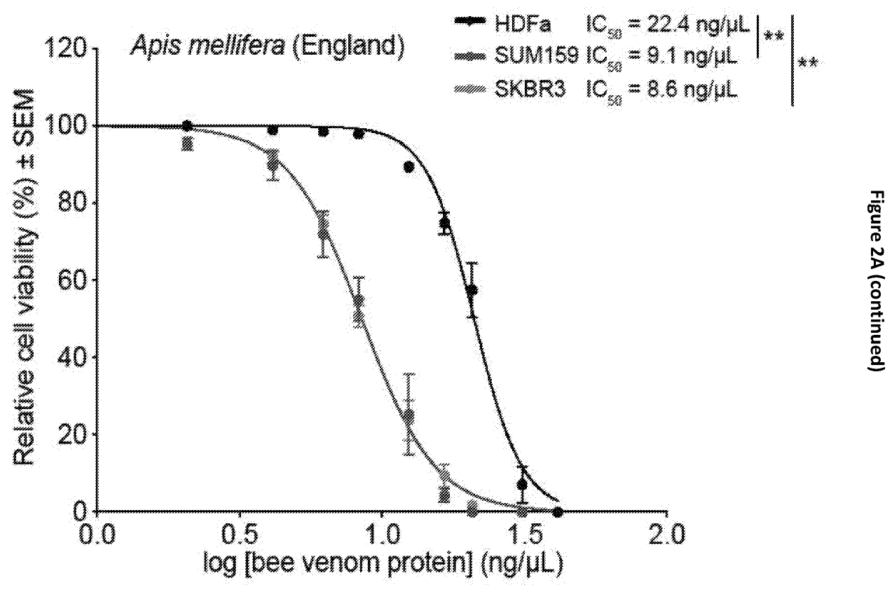

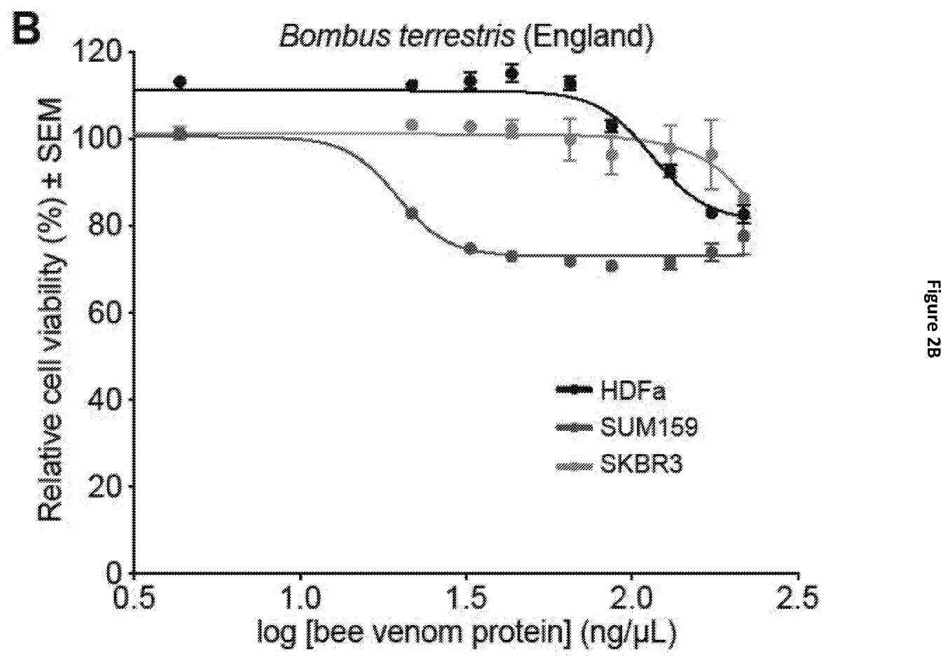

A mouse monoclonal antibody recognizing melittin was then developed to assess the relative abundance of melittin in all honeybee and bumblebee venom samples by ELISA. The relative abundance of melittin was not significantly different across all of the honeybee venom samples from different locations (two-way ANOVA, p>0.999). However, melittin concentrations were significantly higher in honeybee samples compared to bumblebee venom and isotype IgG control (FIG. 3A; two-way ANOVA, p<0.001).

The anticancer effects of melittin were also supported by blocking experiments in vitro, in which the anti-melittin antibody was exploited to rescue cell viability in HDFa and SUM159 cells. Cells were treated with honeybee venom or melittin in combination with increasing concentrations of the anti-melittin antibody. Cell viability was significantly higher when melittin was blocked with the anti-melittin antibody for HDFa and SUM159 cells exposed to honeybee venom or melittin peptide (FIG. 3B; t-tests, p<0.0001). These data suggest that melittin present in honeybee venom is the most prominent bioactive anticancer compound within all the venoms studied.

### Example 2

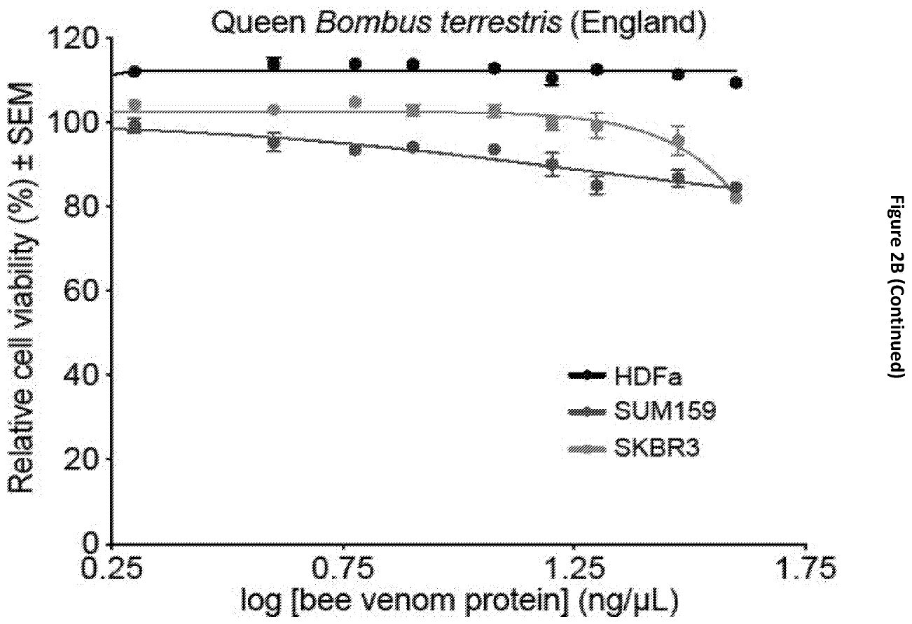

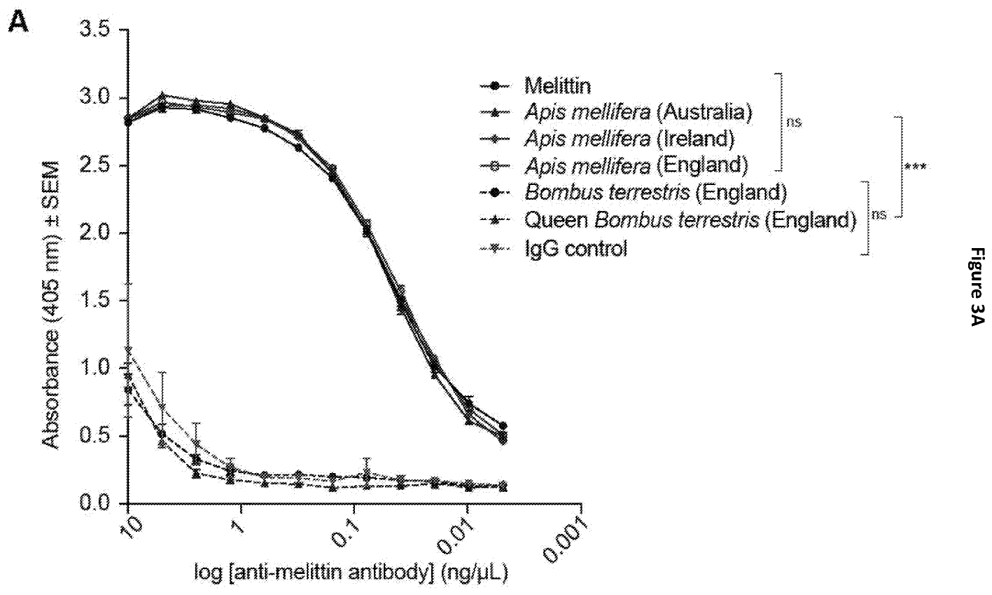

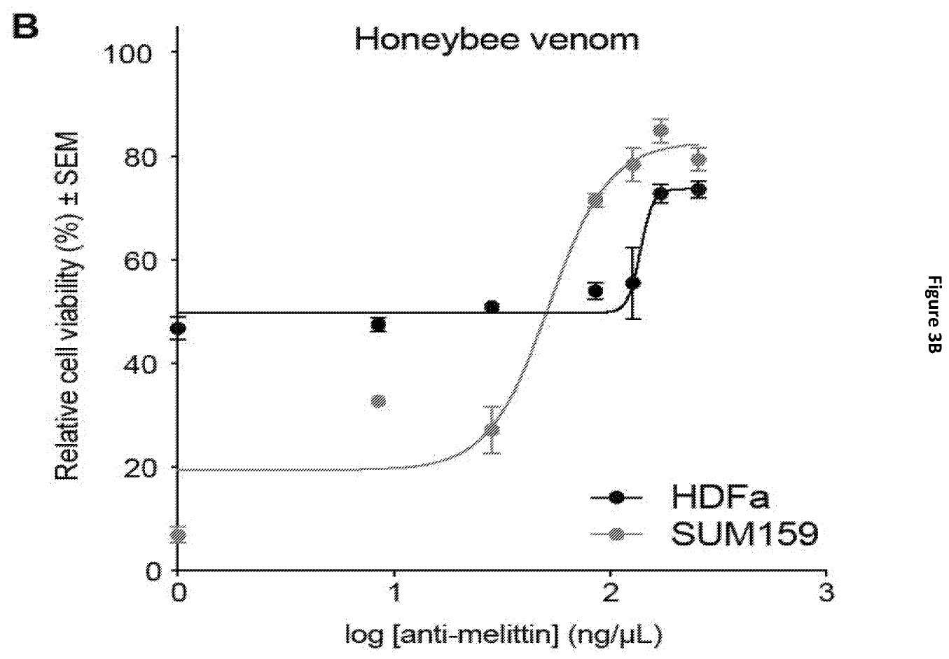

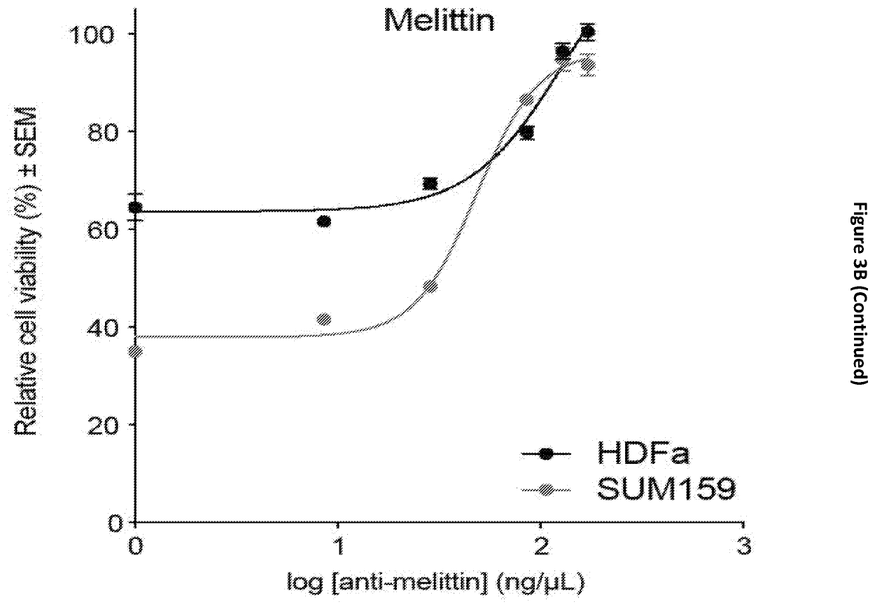

To examine the mechanism and kinetics of cell death, TNBC cells were treated with the IC₅₀ of either honeybee venom or melittin for 18 and 24 hours, and examined with a cleaved caspase-3 assay to quantify apoptotic cell death. Immunoblotting confirmed the induction of cleaved caspase-3 in SUM159 cells, with melittin alone inducing a higher level of apoptosis than honeybee venom at both 18- and 24-hours post-treatment (FIG. 4A).

To quantify the apoptotic, necrotic or dead cell populations after treatment, flow cytometry for the detection of Annexin V (an early marker of apoptosis) was performed. SUM159 cells were treated with either vehicle, honeybee venom, or melittin for 60 minutes (FIG. 4B). Significantly more late apoptotic/necrotic cells were observed for the melittin treated samples (23.6±5.7%) compared to honeybee venom (8.3±1.9%) and vehicle control (4.8±0.4%; two-way ANOVA, p<0.001, mean±SEM). However, there were no significant differences in the levels of early apoptotic or necrotic cells across all conditions (two-way ANOVA, p>0.05, mean±SEM).

To characterize the kinetics of cell death over shorter times, cell viability was measured for HDFa, SKBR3 and SUM159 cells treated for up to one hour with IC₅₀ concentrations of honeybee venom or melittin (FIG. 4C). Honeybee venom rapidly reduced cell viability, with no significant difference between the normal and cancer cell lines over the hour (two-way ANOVA, p=0.97). In contrast, melittin significantly reduced the viability of both breast cancer cell lines compared to the normal cells from 10 minutes onwards, and SUM159 significantly more than SKBR3 from 30 minutes onwards (two-way ANOVA, p<0.0001).

Live cell confocal microscopy (FIG. 4D) and scanning electron microscopy (SEM) (FIG. 4E) in SKBR3 and SUM159 cells illustrated a rapid disruption and shrinking of the plasma membrane with honeybee venom and melittin treatment relative to vehicle control over 10-60 minutes.

### Example 3

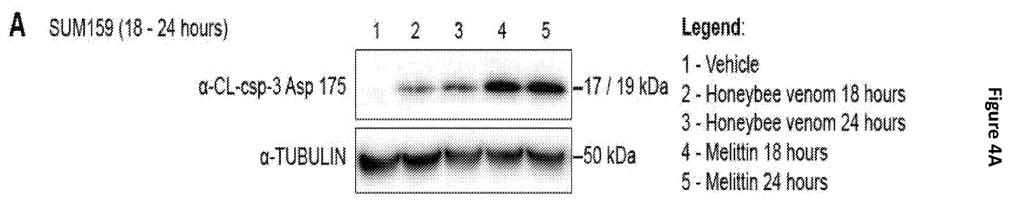

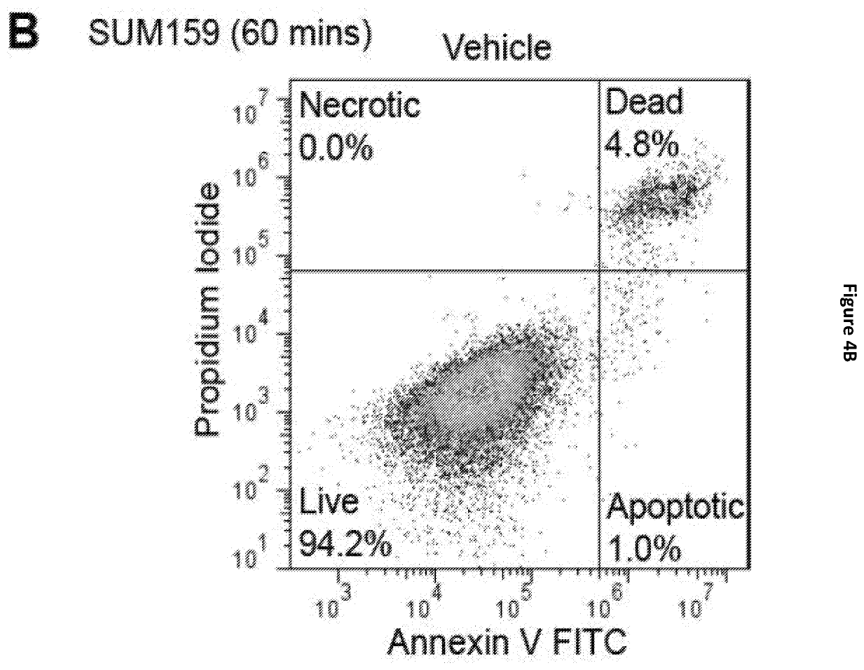

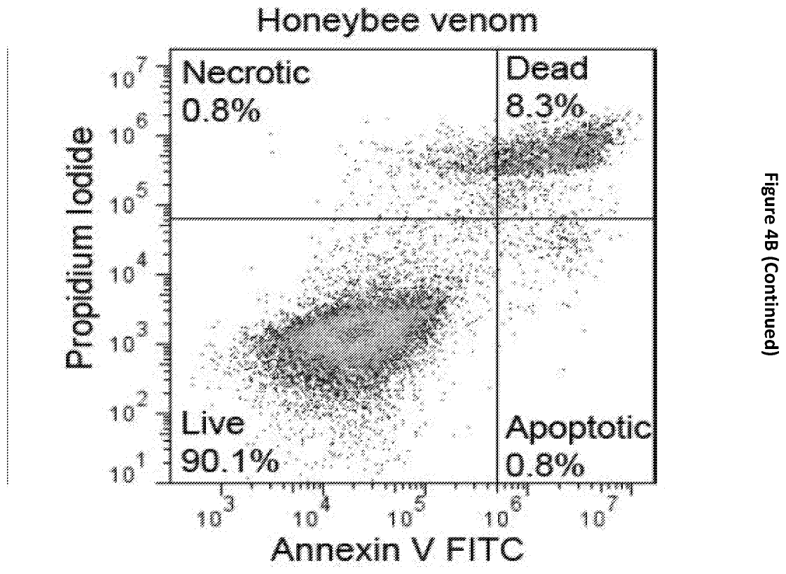

To assess the functional role of the positive (K₂₁RKR₂₄) sequence (SEQ ID NO: 4) in the C-terminus of melittin, a negatively charged melittin peptide (D₂₁EDE₂₄-melittin; SEQ ID NO: 6) was designed. These negative residues were predicted to disrupt the binding of melittin with the plasma membrane. DEDE-melittin did not support any anticancer activity in any of the cell lines tested (FIG. 5A-B). Importantly, the anticancer activity of DEDE-melittin was rescued with a positively charged sequence (K₂₁KKRKV₂₆) (SEQ ID NO: 5) present in the Simian Virus 40 (SV40) large T antigen (peptide SV40-melittin; SEQ ID NO: 9) possessing cell penetrating capacity (FIG. 5B). Similarly, grafting a larger positively charged TAT sequence (transactivated-transcription, derived from HIV-1) in the C-terminus of melittin also restored the activity of DEDE-melittin (peptide TAT-melittin; SEQ ID NO: 10, FIG. 5B). However, the potency of melittin and SV40-melittin were greater than TAT-melittin, which could be due to the larger size of TAT. These data demonstrate that residues required for melittin activity include those residing in the C-terminal α-helix, comprising several key positively charged residues necessary for interaction with the plasma membrane.

### Example 4

To enhance cancer cell selectivity, a bi-functional melittin peptide was generated by engineering an N-terminal alpha helical RGD peptide motif (RGD1-melittin, derived from TGF-β3; SEQ ID NO 2: HGRGDLGRLKK).

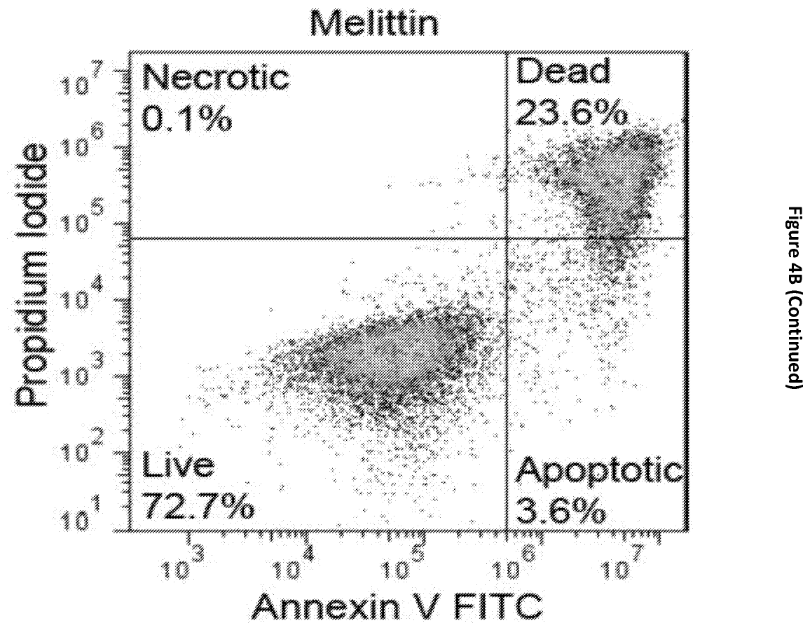

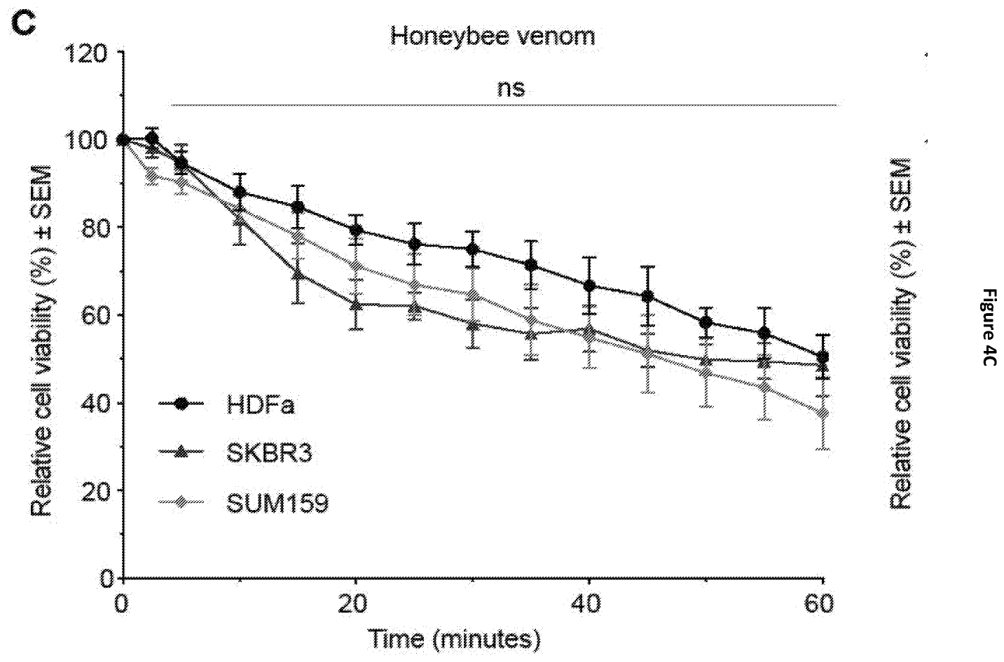

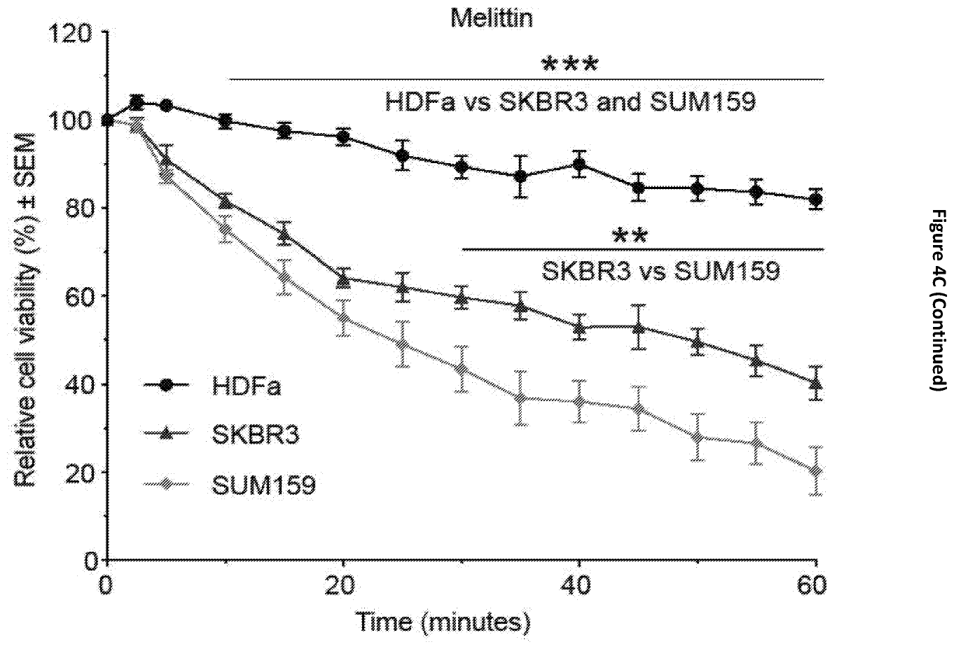

The IC₅₀ of RGD1-melittin was not significantly different compared to parental melittin in T11 cells, indicating that the potency was not affected by the RGD motif (FIG. 5B; t-test, p=0.652). Taking the ratios of the IC₅₀s of HDFa/SUM159 for RGD1-melittin (2.73±0.14) compared to melittin (1.76±0.04), the RGD motif significantly increased the therapeutic window between the normal and TNBC cell lines, showing enhanced cancer cell selectivity conferred by RGD (FIG. 5C; t-test, p<0.01, mean±SEM). Induction of apoptosis in the SUM159 TNBC cells treated with melittin, DEDE-melittin, and RGD1-melittin for 24 hours confirmed the anticancer activity of both melittin and RGD1-melittin, but not DEDE-melittin (FIG. 6A).

The interaction between the anti-melittin antibody and melittin was not significantly different to RGD1-melittin (FIG. 6B; two-way ANOVA, p>0.999), but was significantly different to DEDE-melittin and SV40-melittin (two-way ANOVA, p<0.05), with the absorbance of SV40-melittin not significantly different to IgG control (two-way ANOVA, p>0.1). These data suggested that the monoclonal anti-melittin antibody recognises a conformational epitope that is not disrupted by the engineering of an N-terminal targeting peptide.

Modelling studies indicated that the conformation of the melittin portion of the engineered peptides was not disrupted by either the C-terminal mutation or the N-terminal addition of the RGD motif (FIG. 6C). Each peptide retained the characteristic bent alpha-helix structure potentially facilitating the formation of pores, suggesting that differences in anticancer activity between the mutants is due to electrostatic interactions with the membrane and not gross changes in peptide structure.

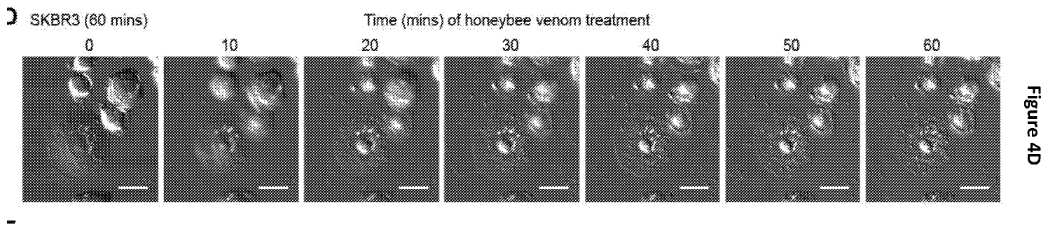

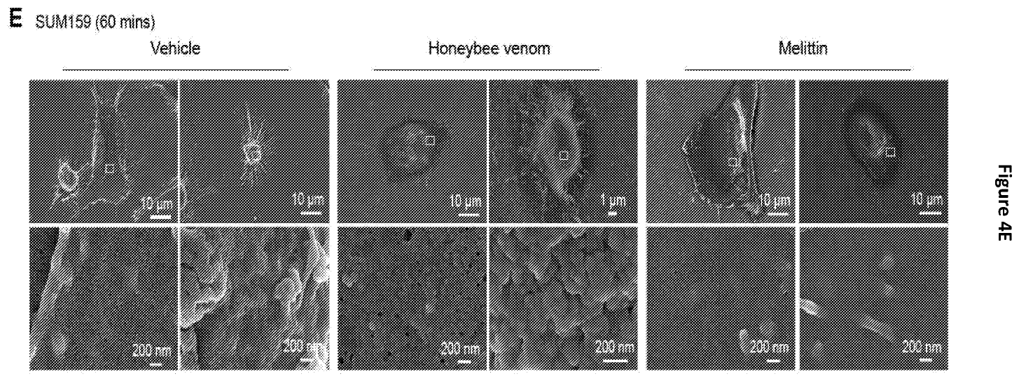

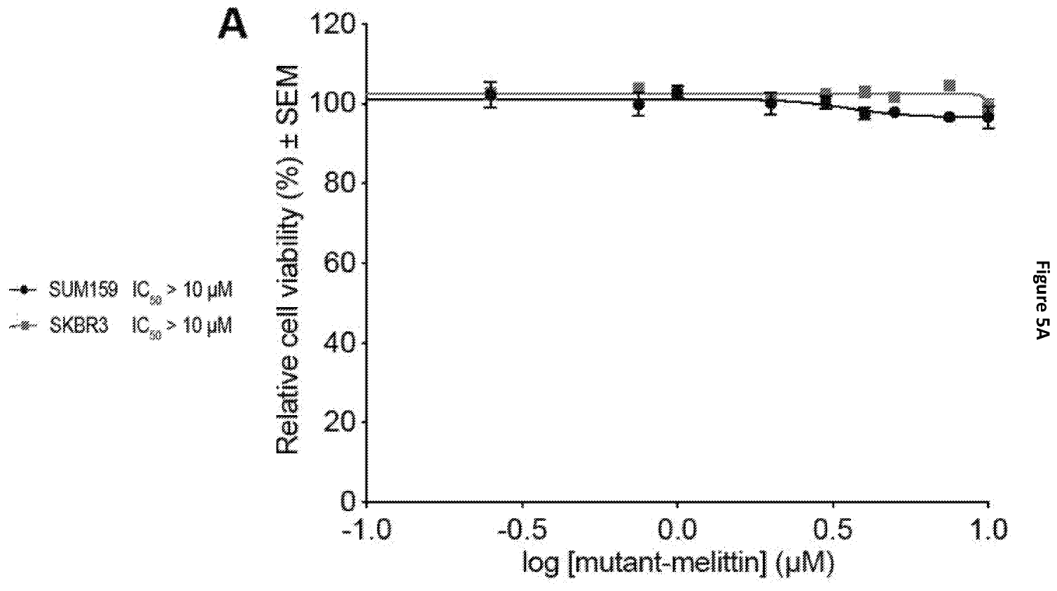

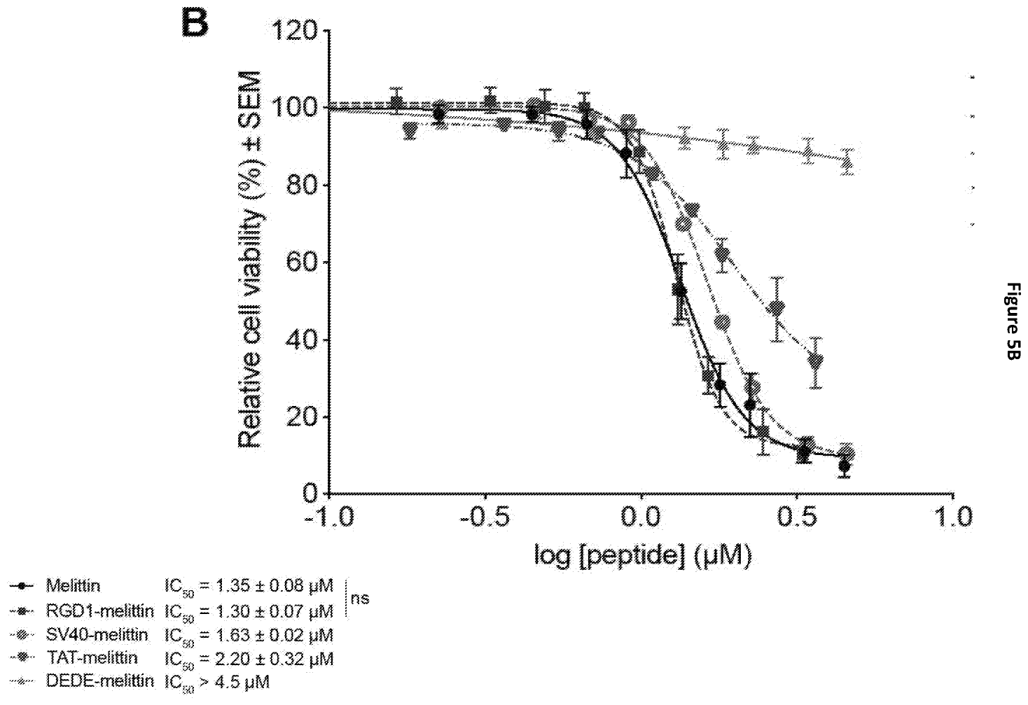

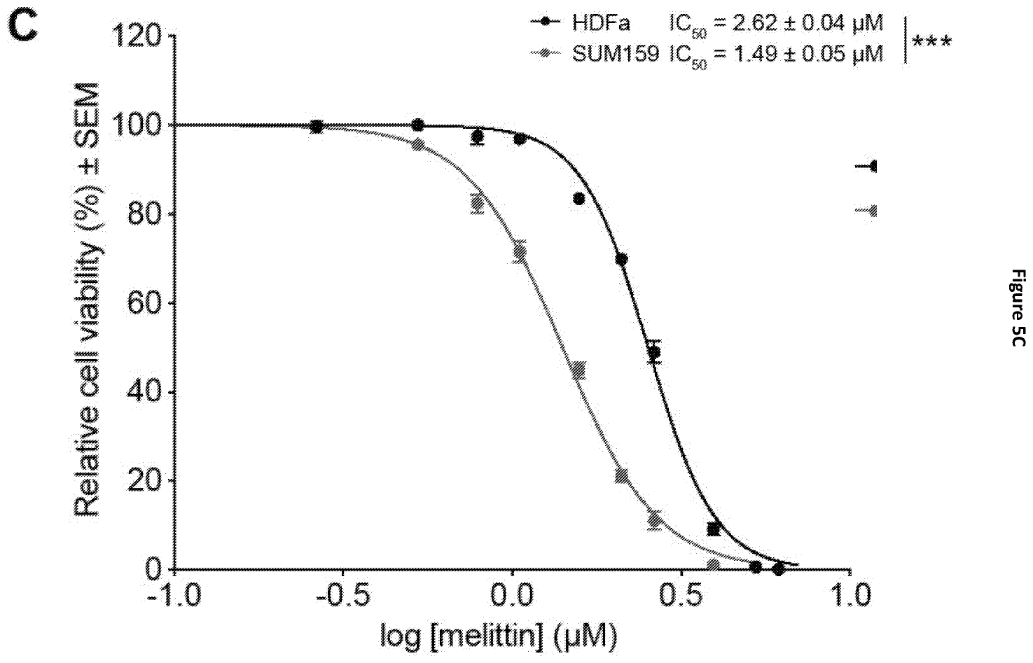

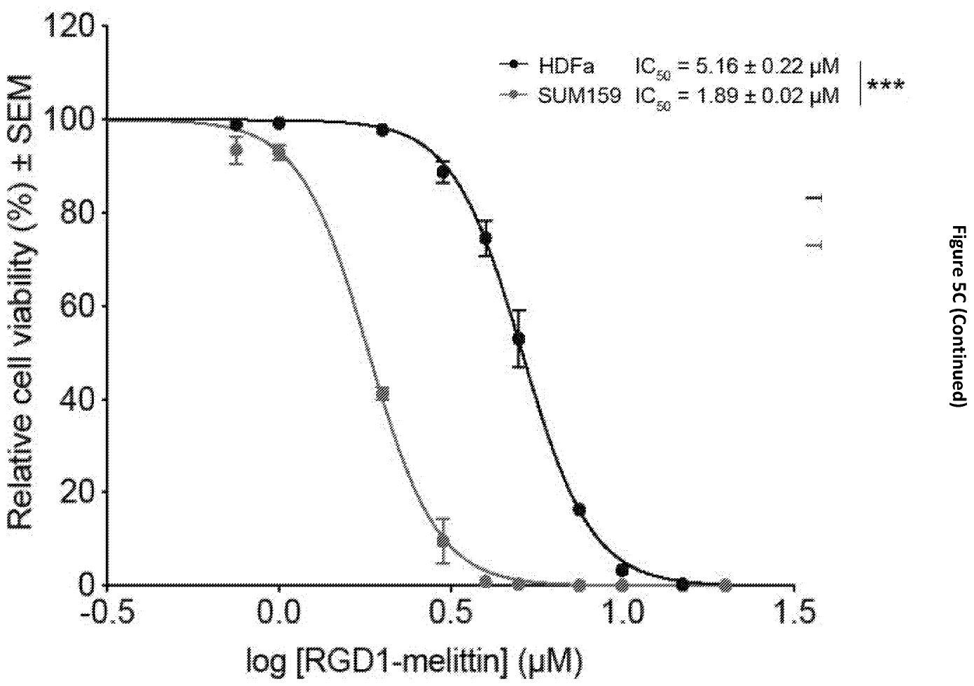

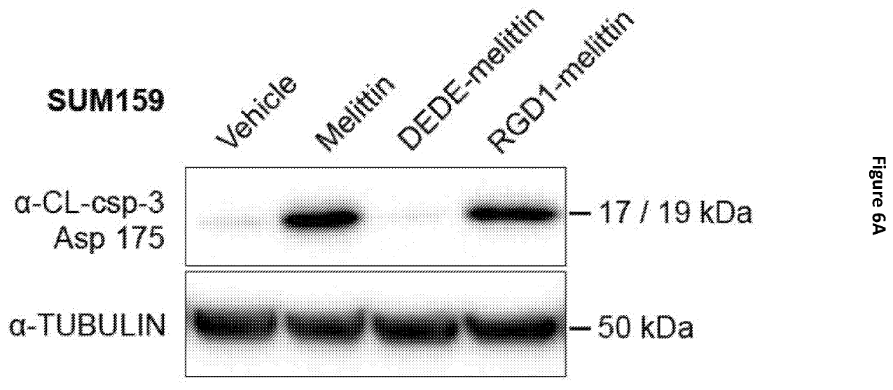

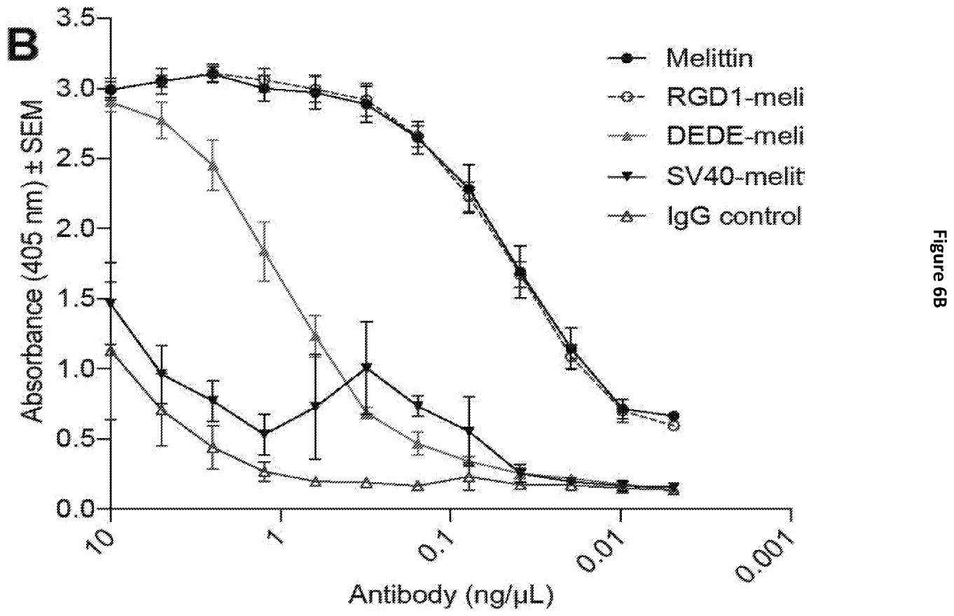

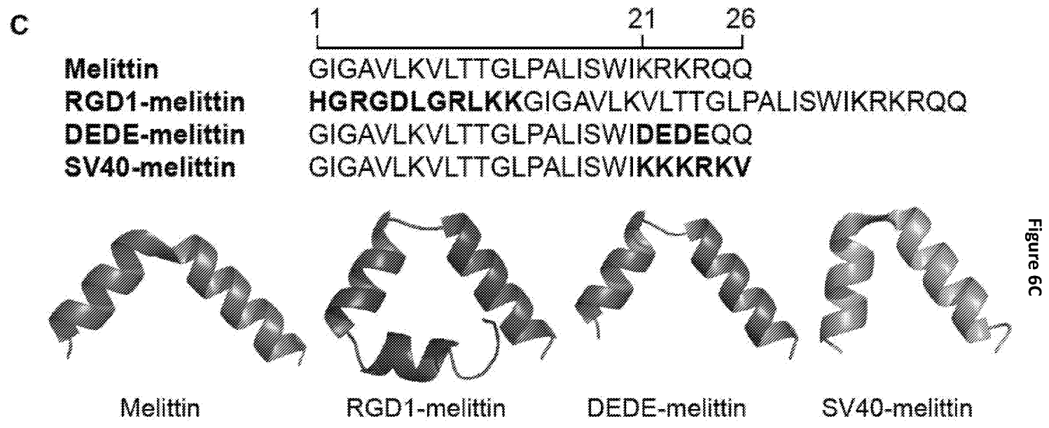

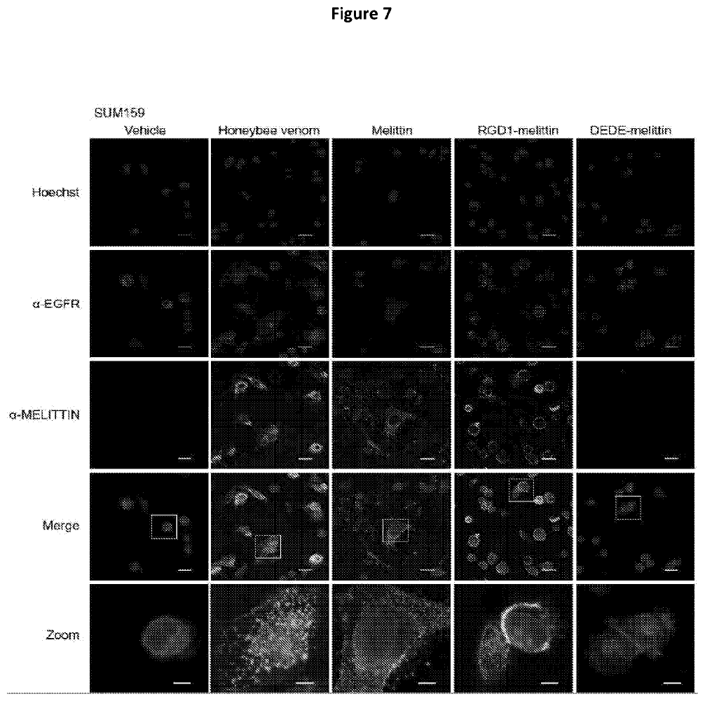

The specificity of the anti-melittin antibody recognizing the active form of melittin was used to detect the subcellular localization of the peptides by immunofluorescence in the melittin sensitive TNBC cell line (SUM159) treated for 30 minutes with either vehicle, honeybee venom, melittin, RGD1-melittin, or DEDE-melittin at IC₅₀ concentrations (FIG. 7). Melittin was found to predominantly localize in the plasma membrane of cells overexpressing EGFR with honeybee venom, melittin, and RGD1-melittin treatments, with a degree of intracellular staining observed for honeybee venom and melittin-treated cells, potentially due to membrane disruption and the formation of endosomes.

Moreover, the pattern of staining of RGD1-melittin appeared distinctively targeted to the plasma membrane alone, which would be in keeping with enhanced selectivity of the targeted peptide for tumour cell surface moieties. A lack of reactivity of the melittin antibody was observed in DEDE-melittin treated cells.

In summary, these results reveal that while the RGD-motif enhances the targeting of melittin to breast cancer cell membranes, the C-terminal positive motif appears important for anticancer activity.

### Example 5

It was then investigated whether both honeybee venom and melittin disrupt RTK-associated signaling pathways by blocking the ligand-dependent activation of EGFR and HER2 in breast carcinoma cells.

To assess this, immunoblotting analysis was conducted on SKBR3 (HER2⁺, EGFR⁺) and SUM159 (EGFR⁺) extracts of cells exposed to EGF and treated with the IC₅₀ of honeybee venom or melittin from 2.5-20 minutes (FIG. 8A-B). Both honeybee venom and melittin down-regulated the phosphorylation of the RTKs and modulated associated PI3K/Akt and MAPK signaling pathways in a time dependent manner.

Treating SKBR3 cells with honeybee venom and melittin strongly downregulated p-HER2 (Tyr1248), p-EGFR (Tyr1068), p-p44/42 MAPK (Thr202/Tyr204), and p-Akt (Ser473 and Thr308) from 5 minutes onwards, with a slight decrease in total HER2, EGFR and Akt protein only after 10 minutes of honeybee venom treatment (FIG. 8A), which could relate to endosome-mediated receptor degradation.

In SUM159, p-EGFR (Tyr1068) was strongly down-regulated by honeybee venom and melittin from 10-20 minutes (FIG. 8B). Treating SUM159 with melittin also suppressed p-Akt (Ser473 and Thr308) at all time points, yet upregulated p-p44/42 MAPK (Thr202/Tyr204) from 10-20 minutes, whereas honeybee venom upregulated p-p44/42 MAPK (Thr202/Tyr204) and p-Akt (Ser473 and Thr308) from 10-20 minutes. The MAPK and Akt pathways may have been upregulated in SUM159 cells due to the release of a negative regulatory feedback loop that triggers ERK signaling to protect the cells from apoptotic cell death. The anti-melittin antibody indicated an increasing amount of melittin present in the lysates of both cell lines over time, with a stronger signal for the melittin treatment compared to honeybee venom in both cell lines.

To characterize the effects on signaling pathways in another TNBC model, immunoblotting was conducted on MDA-MB-231 cells, in which EGF treatment phosphorylated EGFR and induced EGFR expression (FIG. 8C). Melittin reduced the phosphorylation of EGFR and MAPK, downregulating major oncogenic proliferation pathways. Unlike SUM159 cells, EGFR stimulation by EGF did not correlate with an increase in phosphorylation in p-Akt, potentially due to disengagement between EGFR signaling and Akt pathways. Other growth factor receptors such as VEGFR1 may mediate the activation of these pathways.

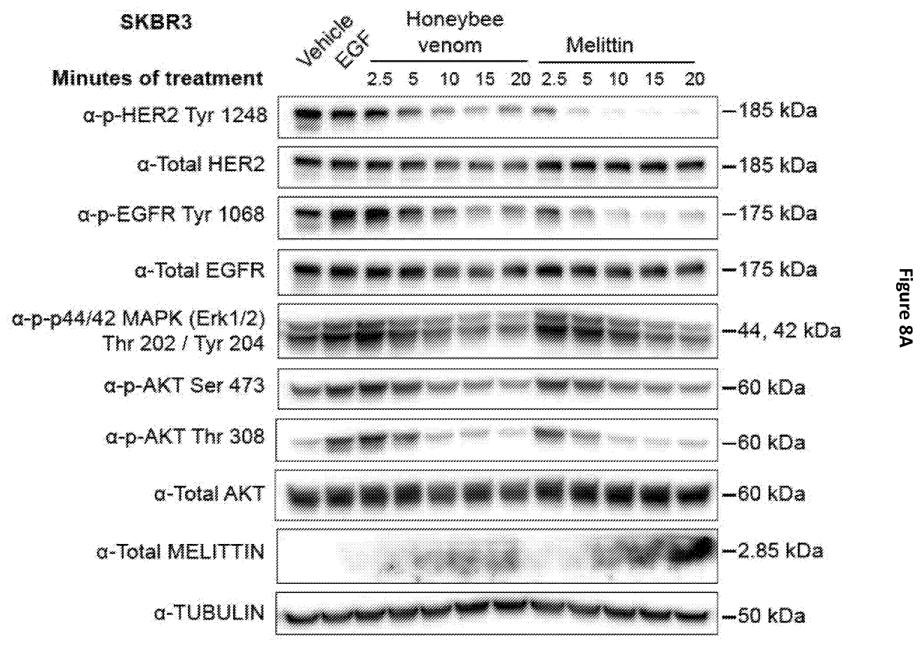

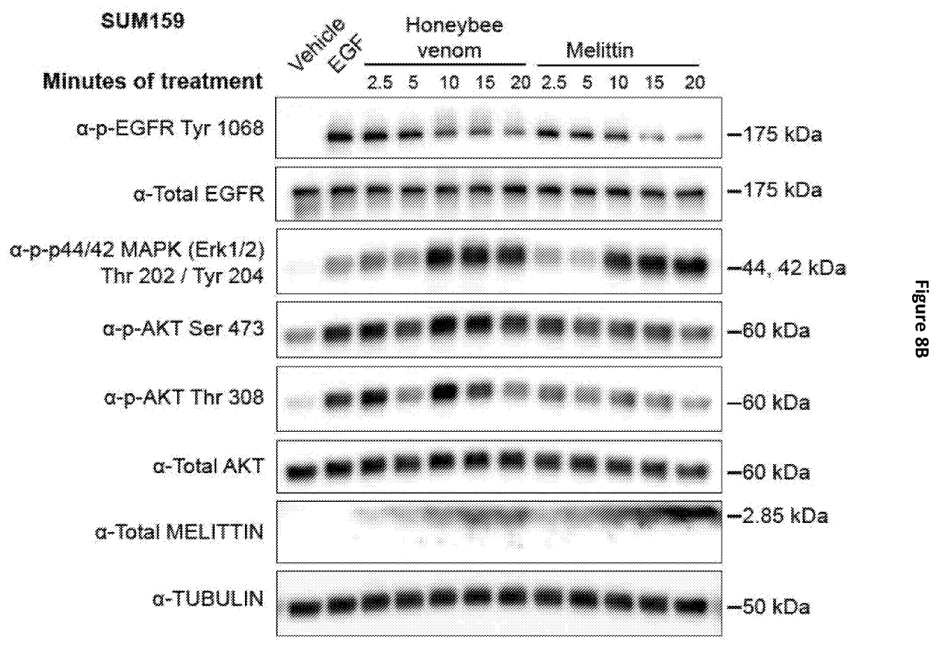

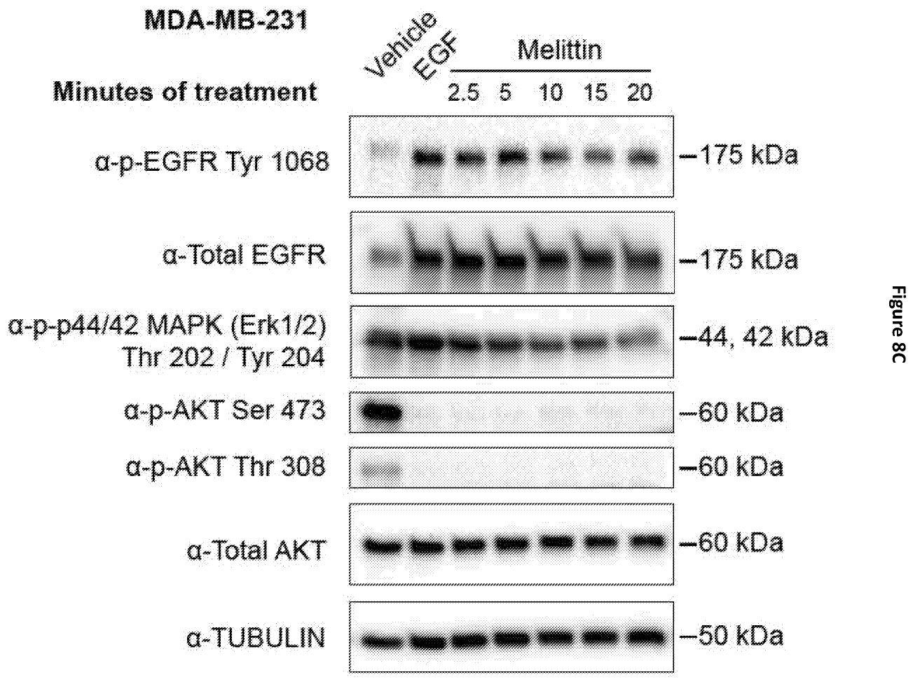

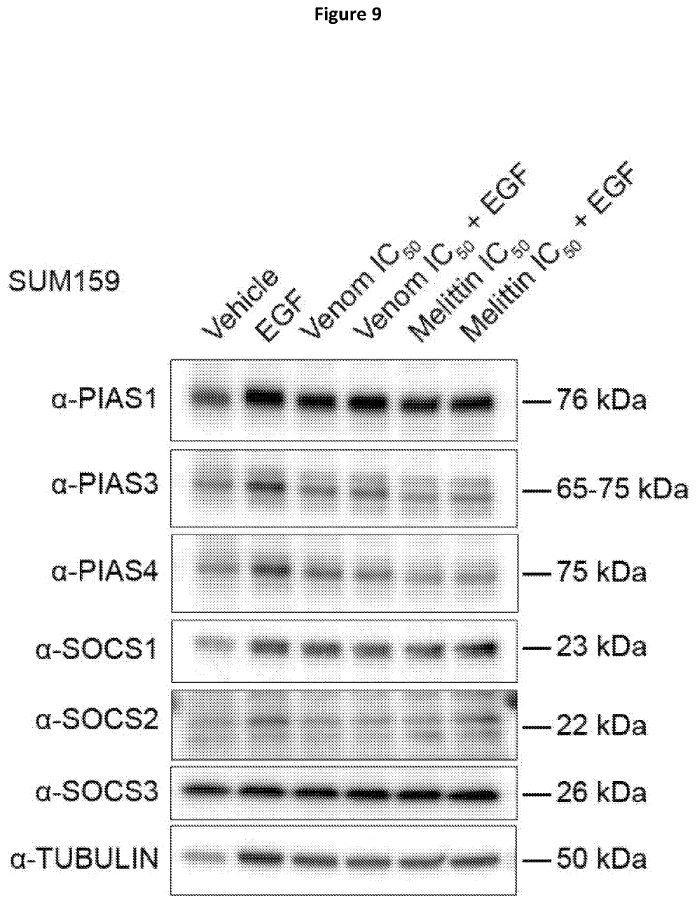

Additionally, the effects of honeybee venom and melittin on the JAK/STAT pathway in SUM159 cells was assessed because melittin has previously been shown to inhibit JAK2/STAT3 signaling in ovarian cancer. No modulatory effects were observed on JAK/STAT pathway inhibitors after a 60-minute treatment with honeybee venom or melittin (FIG. 9).

To determine whether melittin suppressed growth factor receptor phosphorylation by interfering with the binding of RTKs to EGF, bioluminescence resonance energy transfer (BRET) experiments were performed. In this experiment, the NanoLuc reporter was used as the bioluminescent donor molecule and genetically fused to EGFR. Kinetic and saturation BRET experiments were used to monitor the proximity of NanoLuc-EGFR with the fluorescently tagged acceptor molecules TAMRA-EGF (positive control), FITC-melittin, and FITC-DEDE-melittin (negative control) in HEK293FT cells transfected with NanoLuc-EGFR. The BRET signal was determined by monitoring the ratio of light emission from the fluorescent acceptor over the emission from the bioluminescent donor protein, which occurs within a range of less than 10 nm and is indicative of interactions between the tagged molecules of interest.

A range of concentrations of each peptide were selected including the IC₅₀ of FITC-melittin, with the corresponding molar concentrations of FITC-DEDE-melittin. It was found that the BRET signal increased in a dose-dependent manner for TAMRA-EGF and FITC-DEDE-melittin, and to a lesser extent FITC-melittin (FIG. 10A-C). A non-specific peptide designed against the Engrailed 1 (EN1) transcription factor (FITC-EN1-mutant) exhibited similar BRET ratios and kinetics to FITC-DEDE-melittin (FIG. 10D), indicating that further experiments were required to ascertain the specificity of the binding interactions with EGFR.

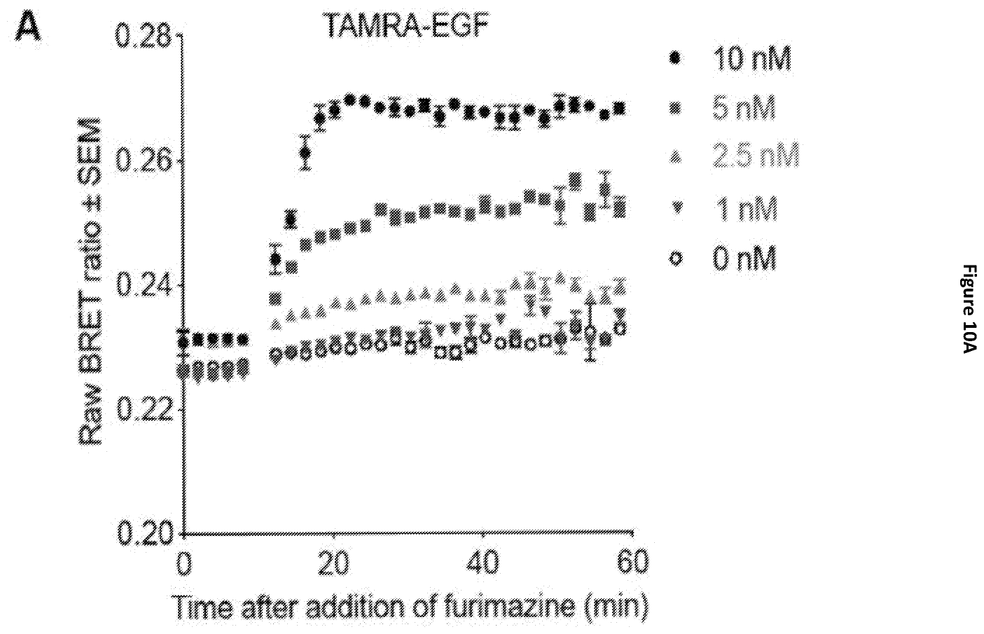

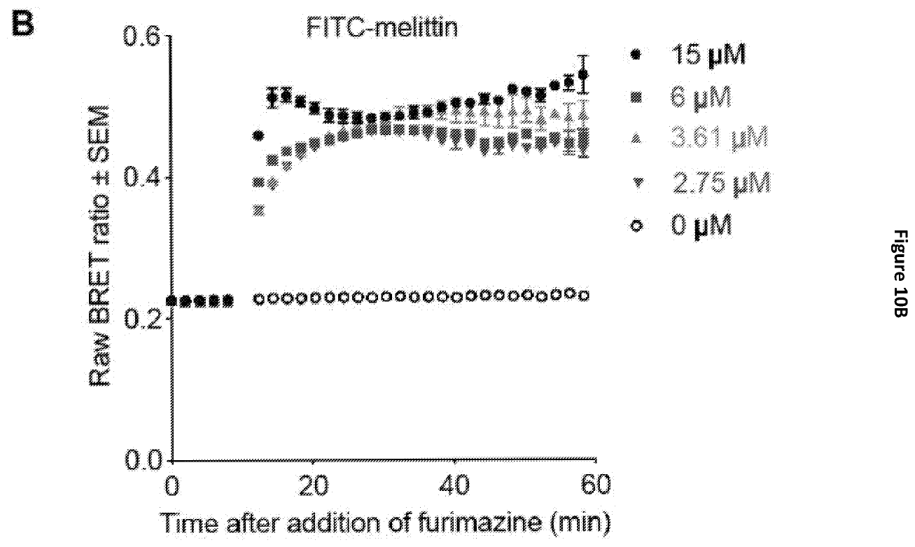

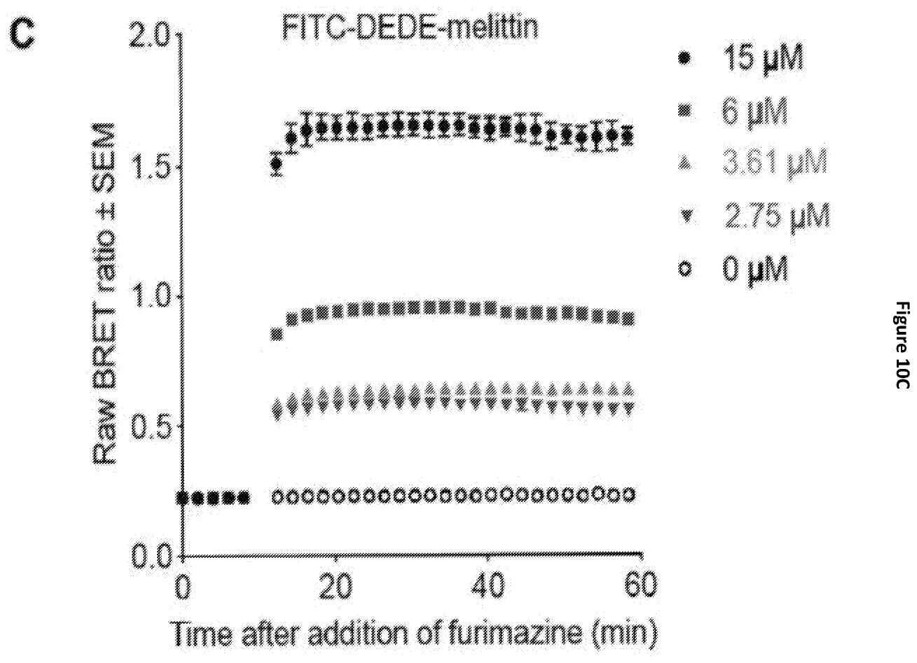

To determine whether melittin bound to EGFR at the EGF binding site, saturation BRET assays were conducted to assess the competition of EGF with each of the peptides binding to NanoLuc-EGFR. While the binding of TAMRA-EGF to NanoLuc-EGFR was saturable and significantly reduced in the presence of 1 μM EGF (FIG. 11A; two-way ANOVA, p<0.0001), the BRET signals of FITC-melittin and FITC-DEDE-melittin were not saturable and not significantly different with or without 1 μM EGF (FIG. 11B-C; two-way ANOVAs, p>0.999), suggesting that neither melittin nor DEDE-melittin bound at the EGF binding site.

Overall, and without wishing to be bound by theory, these data support a model in which melittin becomes incorporated into the plasma membrane of cancer cells via a charged sequence present in the C-terminus, inducing plasma membrane remodeling and disruption. BRET data indicates that melittin may be positioned at a distance within 10 nm from the RTKs without interfering with the endogenous growth factor binding site (FIG. 11).

### Example 6

Melittin forms transmembrane pores of approximately 4.4 nm diameter, which may facilitate the passive entrance of chemotherapeutics into cancer cells. Whether melittin could synergize with chemotherapeutic agents to achieve more potent therapeutic efficacy was therefore explored.

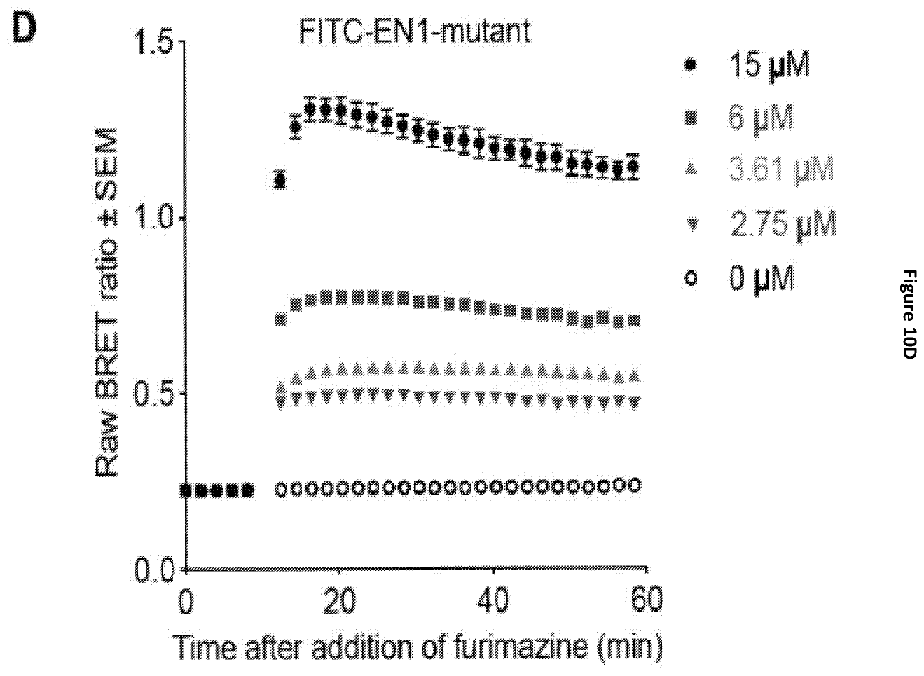

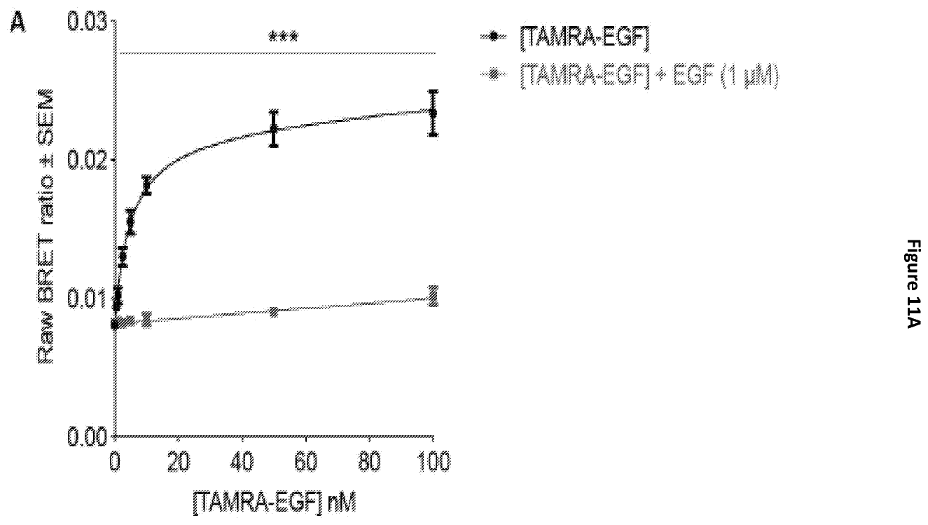

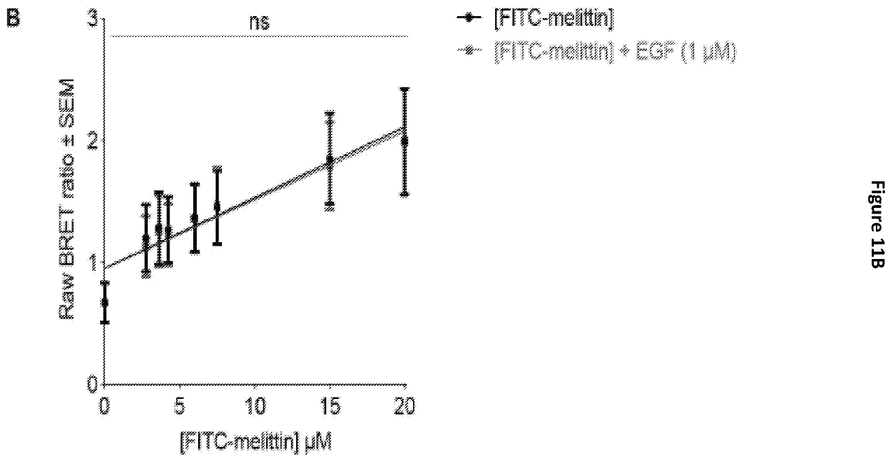

The murine p53⁻ TNBC cell line T11 was treated with docetaxel or cisplatin in combination with either honeybee venom or melittin, and cell viability assays were conducted to determine the combination index (CI) between the treatments (FIG. 12A). A Combination Index less than 1 was observed for all the concentrations tested indicating strong synergistic interactions (FIG. 12B).

To investigate the efficacy of the combination of melittin and docetaxel in reducing TNBC growth in vivo, T11 cells were transplanted in BALB/c mice. This allograft model recapitulates highly aggressive, TNBC claudin-low disease in mice with an intact immune system. Three days after the generation of T11 tumours (˜50 mm³), mice were randomized into four groups (n=12 mice/group) and treated intratumorally with either vehicle, melittin (5 mg/Kg), docetaxel (7 mg/Kg), or a combination of melittin (5 mg/Kg) and docetaxel (7 mg/Kg). Mice were treated every two days from day three, with a total of seven treatments.

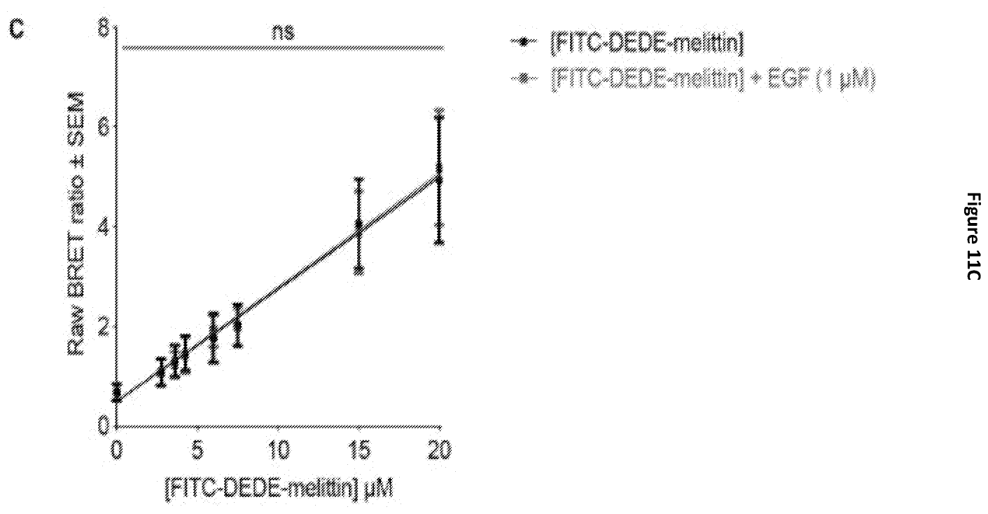

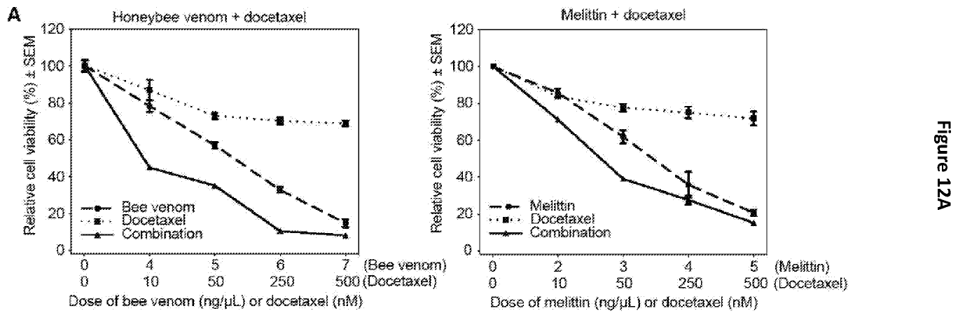

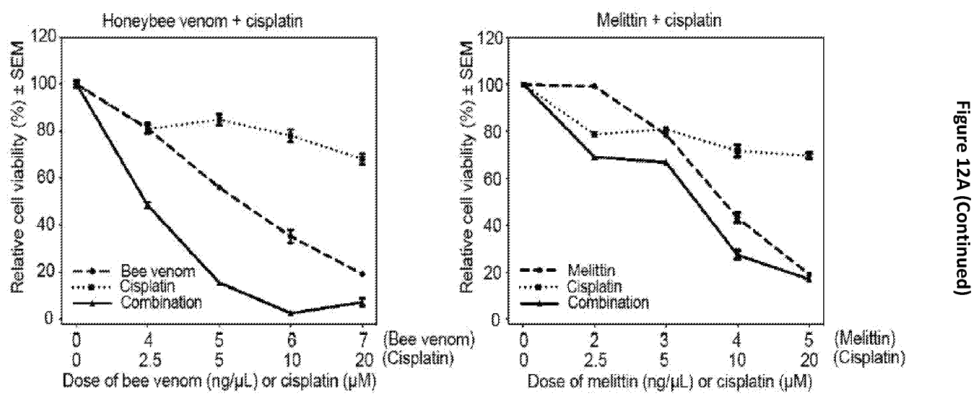

Tumour control for the combination treatment was superior compared to either treatment alone or vehicle, particularly on days 7 and 9 post-inoculation of T11 cells, with the combination achieving a significant reduction in tumour volume (FIG. 13A, one-way ANOVAs, p<0.001). This suggests that tumours resistant to docetaxel could be rendered sensitive by the addition of melittin. These studies were validated by bioluminescence imaging (BLI) to non-invasively track changes in in vivo tumour growth in T11 cells tagged with a luciferase-containing construct (FIG. 13B). Here again, improved tumour control was found for the docetaxel and melittin combination treatment at days 10, 12 and 14 compared to all other groups.

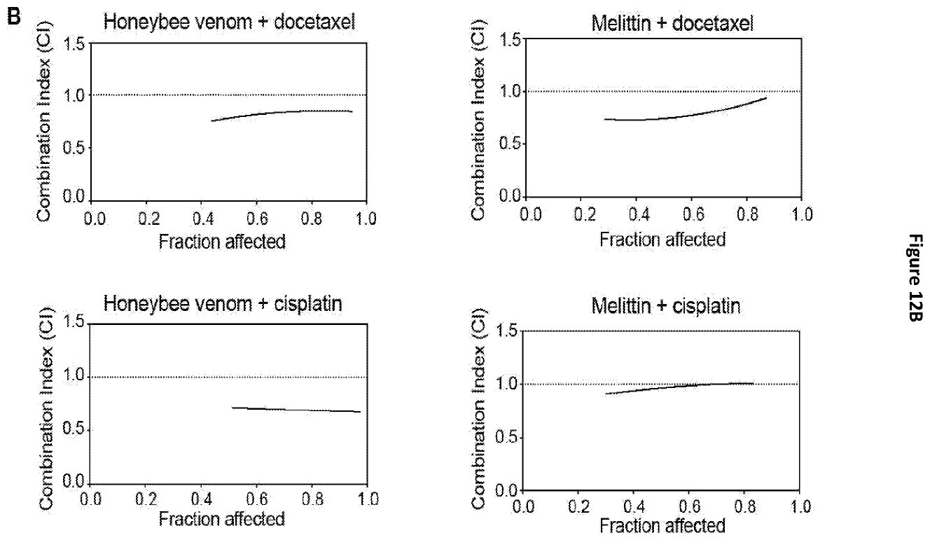

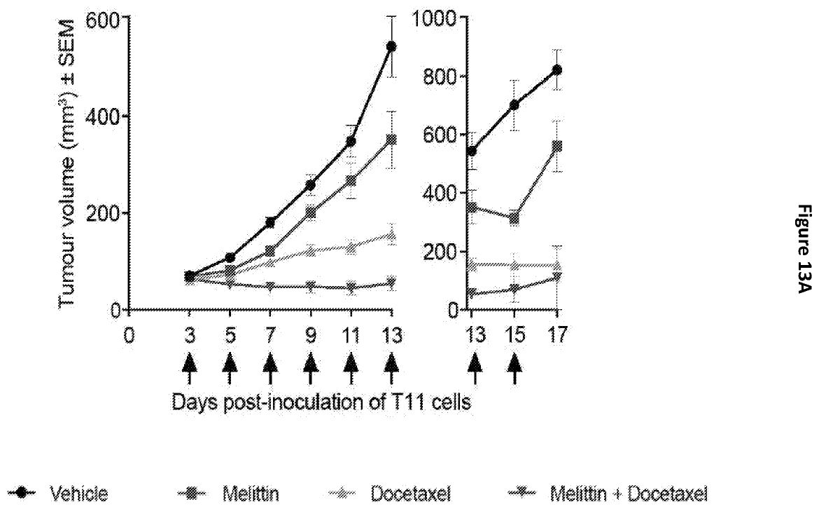

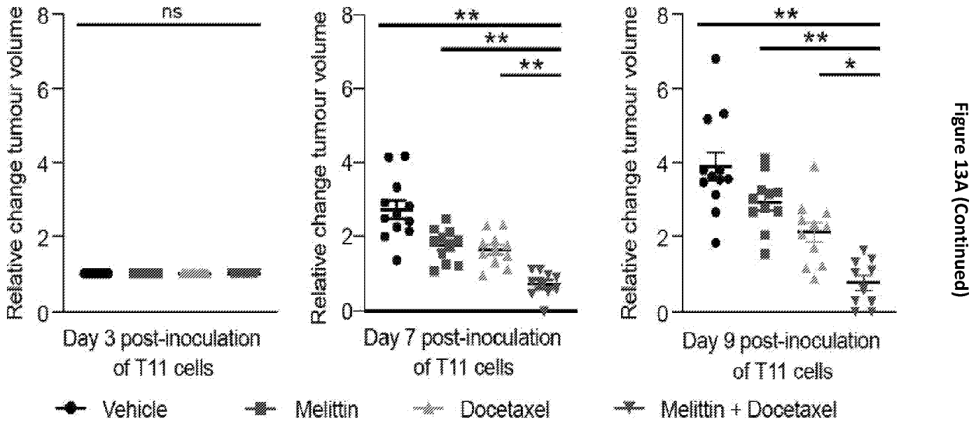

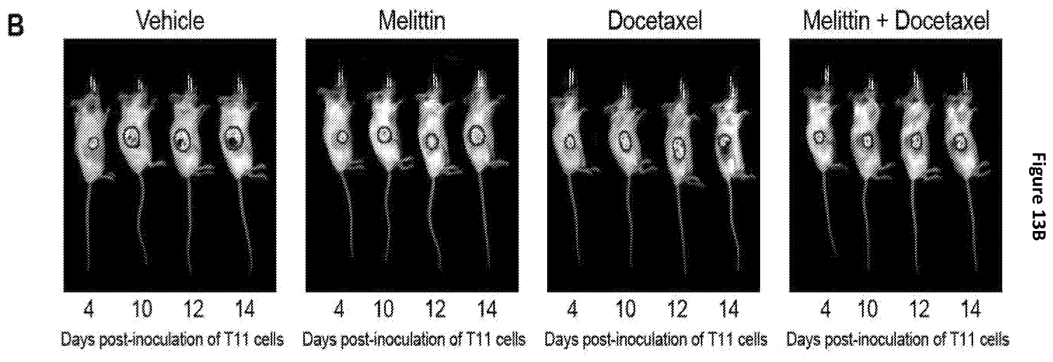

The therapeutic effects of melittin and docetaxel were validated in tumour tissues at day 14 post-inoculation of T11 cells by immunohistochemistry and immunofluorescence (FIG. 14A). The anti-melittin antibody confirmed the intra-tumoral localization of melittin positive cells in both the melittin treatment group (61.9±0.7%), and the combination treatment group (55.8±1.3%), and not in vehicle control (one-way ANOVA, p<0.001, mean±SEM). A significant reduction in tumour cell proliferation (as assessed by Ki-67 expression) was found in the tumours treated with the combination of melittin and docetaxel (5.7±0.8%) relative to vehicle (59.8±1.7%), compared to either melittin (31.7±1.3%), or docetaxel alone (21.0±1.3%; one-way ANOVA, p<0.01, mean±SEM). TUNEL staining confirmed a significantly higher DNA fragmentation and apoptosis induction in the combination group (81.0±3.1%) compared to vehicle (1.0±0.4%; one-way ANOVA, p<0.01, mean±SEM).

Interestingly, docetaxel did not affect the levels of the immune checkpoint protein programmed death ligand-1 (PD-L1) in the tumours. PD-L1 reduces the functionality of activated T cells. Consequently, immune checkpoint blockades in combination with chemotherapy prevents T-cell PD-L1 recognition preventing this adaptive immune resistance in TNBC and thereby increasing therapeutic efficacy over chemotherapy alone.

In contrast to docetaxel alone (84.3±0.6%), melittin significantly reduced PD-L1 expression in tumours when used alone (52.9±2.4%) or with the combination (44.3±4.2%) compared to vehicle (84.9±1.6%; one-way ANOVA, p<0.01, mean±SEM).

In summary, these studies support the notion that melittin sensitizes T11 cells to docetaxel treatment, and that melittin could help attenuate the expression of immune-checkpoint proteins, consequently improving anti-tumoral immune responses.

Immunohistochemistry was then performed in the treated T11 tumours to detect p-HER2 (Tyr1248) and p-EGFR (Tyr1068) (FIG. 14B). The expression of EGFR was moderately but significantly reduced by melittin and docetaxel combination (75.8±6.4%) compared to vehicle (100.0±9.1%; one-way ANOVA, p<0.05, mean±SEM). The expression of HER2 was not significantly different across all treatment groups (one-way ANOVA, p=0.1536). For p-EGFR (Tyr1068), the phosphorylation was reduced to a significantly lower level by melittin and docetaxel combination treatment (9.0±2.4%) compared to vehicle (100.0±8.1%; one-way ANOVA, p<0.0001, mean±SEM). The levels of p-HER2 (Tyr1248) were also reduced to a significantly lower level in the melittin and docetaxel combination treatment (50.3±7.8%) compared to vehicle (100.0±5.6%; one-way ANOVA, p<0.0001, mean±SEM). The decrease in EGFR and HER2 phosphorylation in vivo after melittin treatment is consistent with the observed effects of melittin in reducing the phosphorylation of these RTKs in SKBR3, SUM159, and MDA-MB-231 cells (FIG. 8).

### Example 7

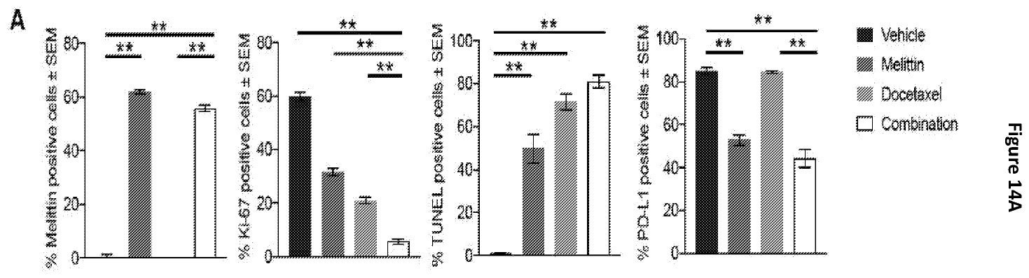

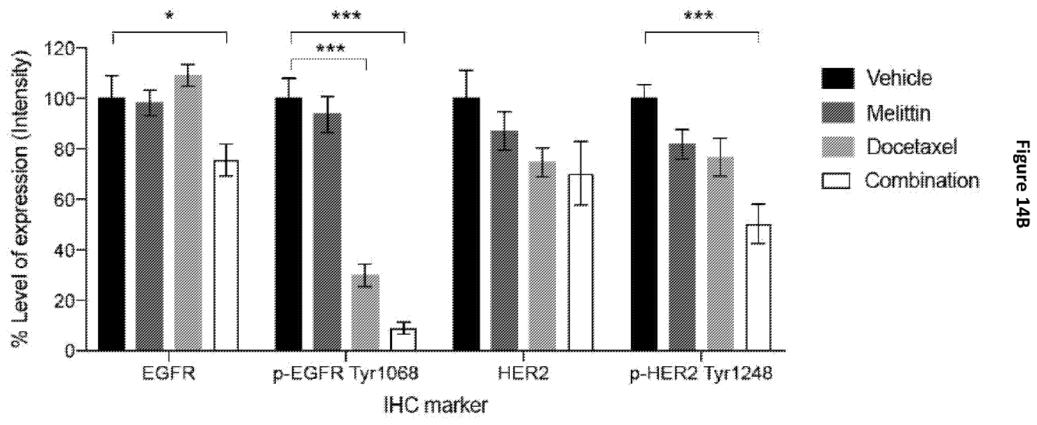

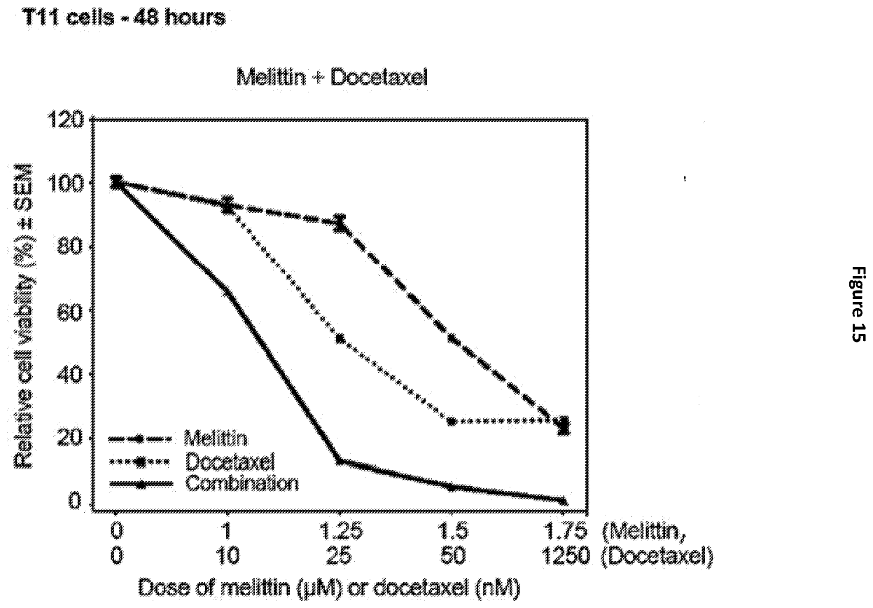

The murine p53 TNBC cell line T11 was treated with docetaxel in combination with either melittin or RGD1-melittin, and cell viability assays were conducted to determine the combination effect between the treatments (FIG. 15). Both melittin and RGD1-melittin had a beneficial effect when combined with docetaxel in vitro. The combination of RGD1-melittin and docetaxel reduced tumour cell viability more effectively than either RGD-1 melittin or docetaxel alone.

### Example 8

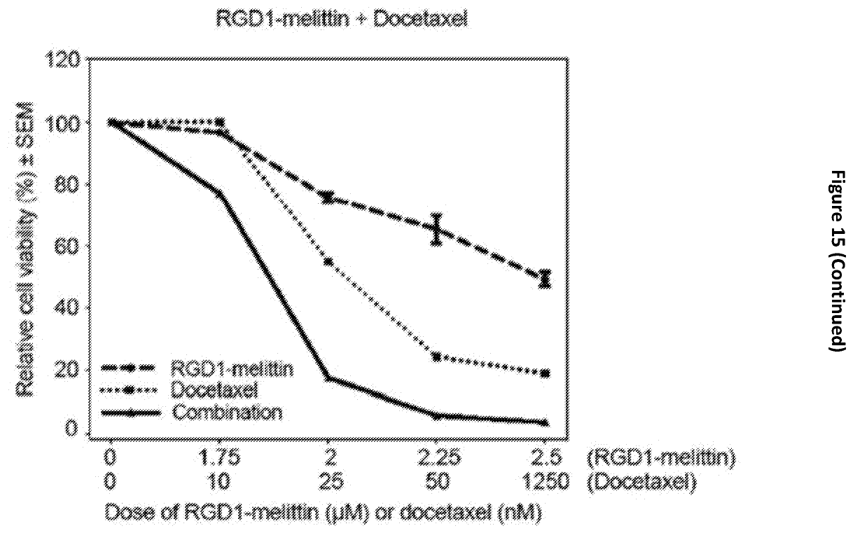

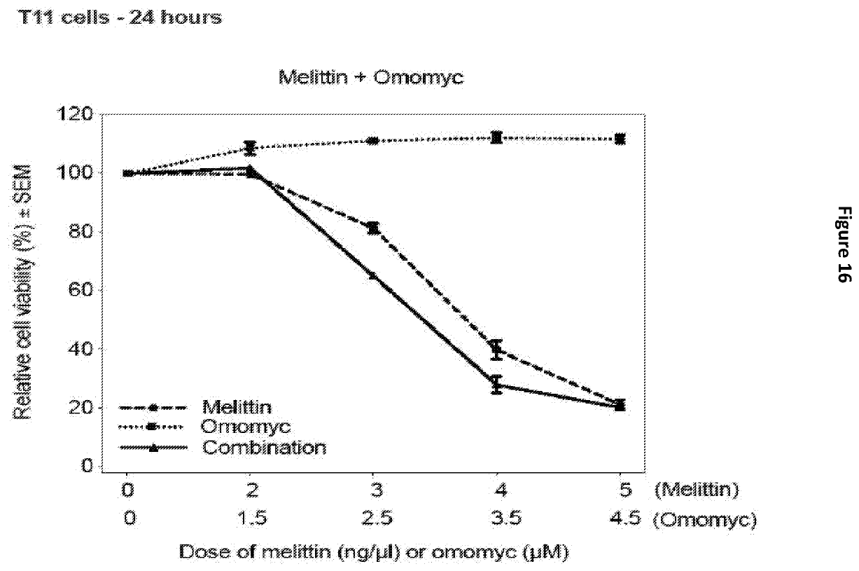

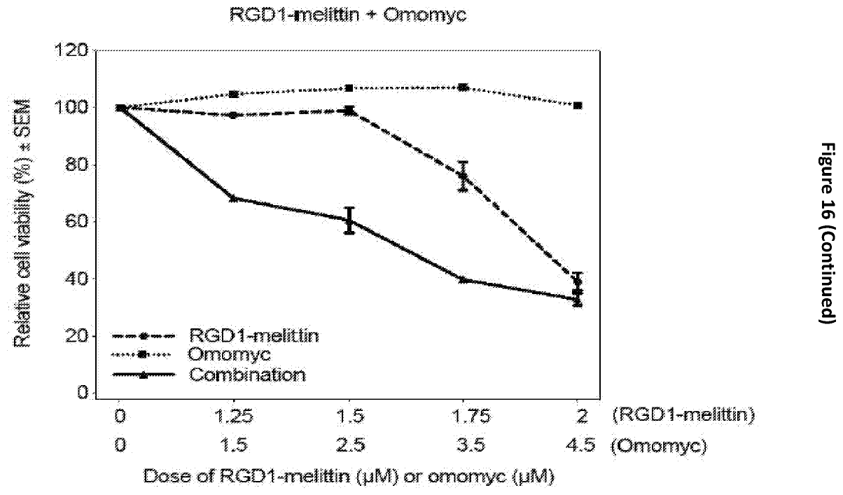

The murine p53 TNBC cell line T11 was treated with omomyc in combination with either melittin or RGD1-melittin, and cell viability assays were conducted to determine the combination effect between the treatments (FIG. 16). Both melittin and RGD1-melittin had a beneficial effect when combined with omomyc in vitro. The combination of RGD1-melittin and omomyc reduced tumour cell viability more effectively than either RGD-1 melittin or omomyc alone.

### Example 9

The OVCAR3 and COV362 ovarian cancer cell lines were treated with active RGD1-melittin and mutant RGD1-melittin (DEDE-RGD1-melittin). The COV362 cells were cultured in DMEM low glucose medium supplemented with 10% FBS. The OVCAR3 cells were grown in RPMI medium supplemented with 20% FBS. Cells were seeded overnight at a density of 5000 cells/p96-well plate and treated with active RGD1-melittin and mutant RGD1-melittin (RGD1-DEDE melittin), for 24 h at concentrations up to 20 M. After treatments, cell viability was assessed using the CellTiter-Glo® assay according to the manufacturer's protocol (Promega; NSW, Australia). Luminescence signals were measured using the EnVision Multilabel Plate Reader (PerkinsElmer Inc.; Waltham, MA, USA). IC50s were calculated and transformed 95% confidence intervals provided by GraphPad Prism 6 software analysis (GraphPad Software Inc., San Diego, CA, USA).

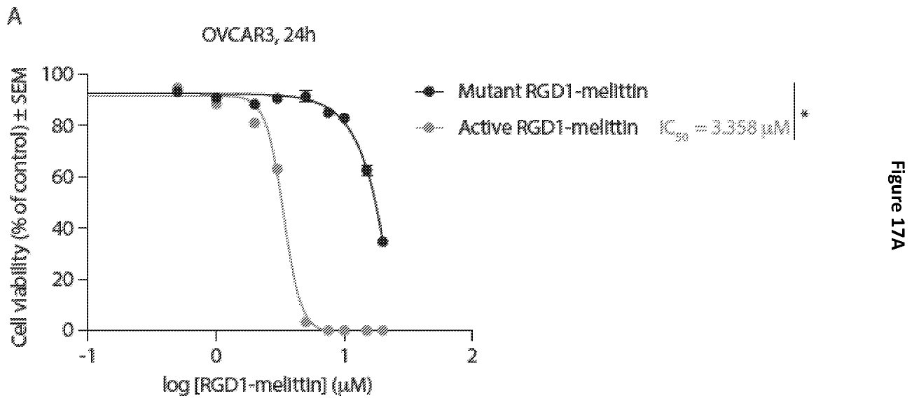

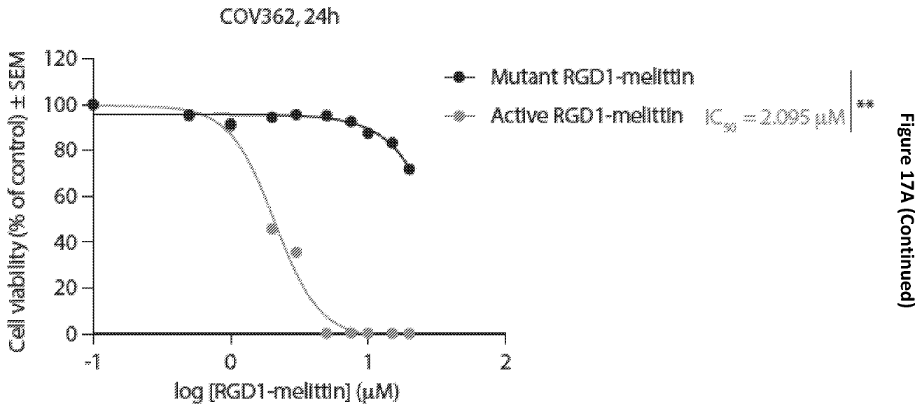

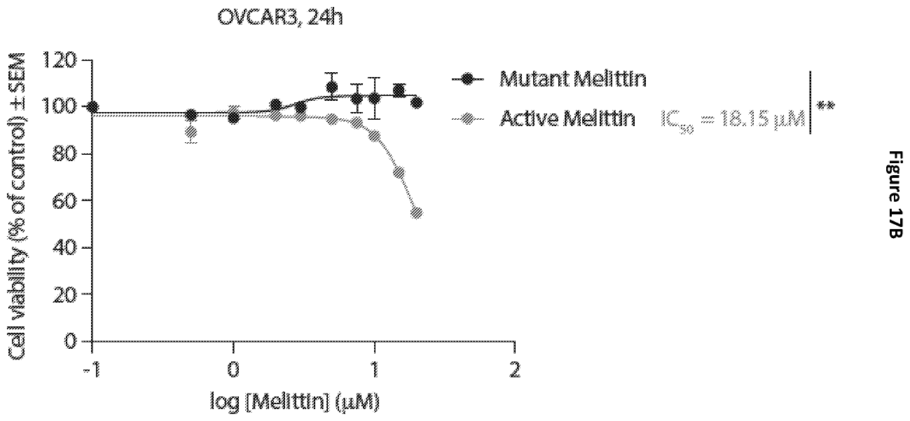

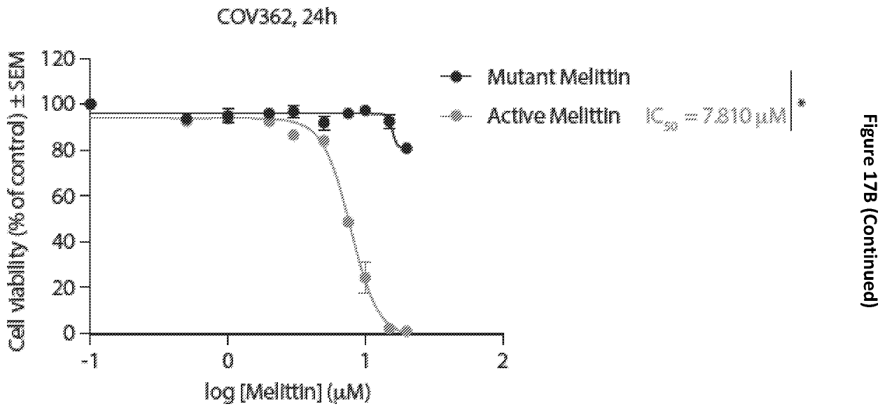

The results showed that the active RGD1-melittin peptide was significantly more selective for highly aggressive ovarian cancers compared with the inactive mutant RGD1-melittin (p<0.005). Further, the active RGD1-melittin was significantly more selective for ovarian cancer than melittin. The IC₅₀ for active RGD1-melittin for the OVCAR3 cell line was 3.358 μM, compared with 18.15 μM for active melittin (approximately 6 fold lower), whereas IC₅₀ for active RGD1-melittin for the COV362 cell line was 2.095 μM, compared with 7.810 μM for active melittin (approximately 3.7 fold lower). The results of the cell viability assays are provided in FIG. 17.

### Example 10

The expression of PD-L1 of TNBC cells (T11 and SUM159) and HER2-enriched breast cancer cells (SKBR3) treated with active RGD1-melittin and mutant RGD1-melittin (DEDE RGD1-melittin) was measured using immunofluorescence.

Cells were seeded overnight on coverslips at a density of 3×105 in a p24-well plate coated with poly-L-lysine (Sigma-Aldrich) for 20 min and then washed twice with purified water. Cells were treated with RGD1-melittin and mutant RGD1-melittin for 24 h at IC50 concentrations. The following day, cells were fixed with 4% paraformaldehyde for 20 min, washed twice with PBS, blocked with 5% normal goat serum for 1 h at room temperature and separately incubated with a-Ki-67 (Cell Signaling Technology #9449, 1:500) and a-PD-L1 (Abcam #238697; 1:500) in antibody diluent (1% BSA and 0.3% Triton X-100 in PBS) overnight at 4° C. The cells were washed three times with PBS and then incubated with a goat α-mouse secondary Alexa Fluor 488-conjugated antibody (Thermo Fisher Scientific #A11001, 1:500) and Hoechst-33258 (1:5000) in PBS for 1 h at room temperature. After three more washes of PBS, the coverslip containing cells were mounted onto glass coverslips with SlowFade Diamond Antifade Mountant (Thermo Fisher Scientific). Slides were imaged using the confocal fluorescence Nikon Ti inverted microscope. Images were taken at 40× magnification and processed using ImageJ.

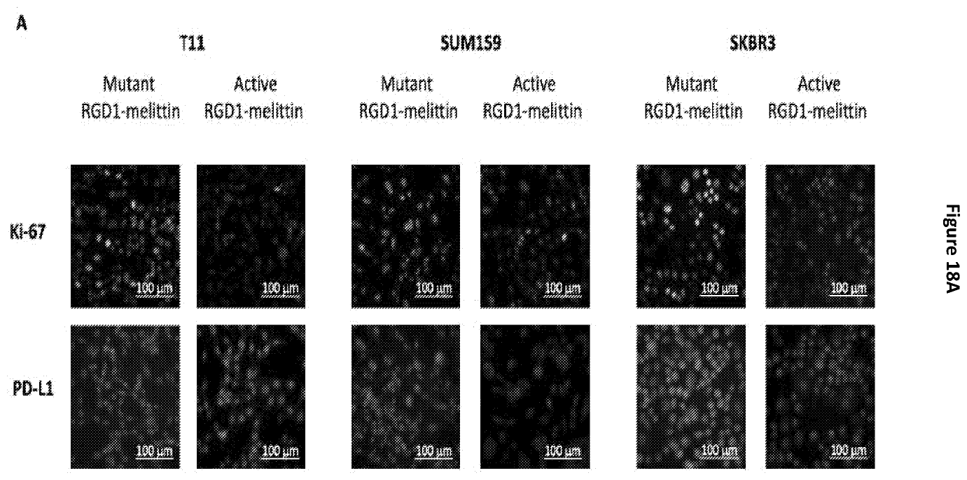

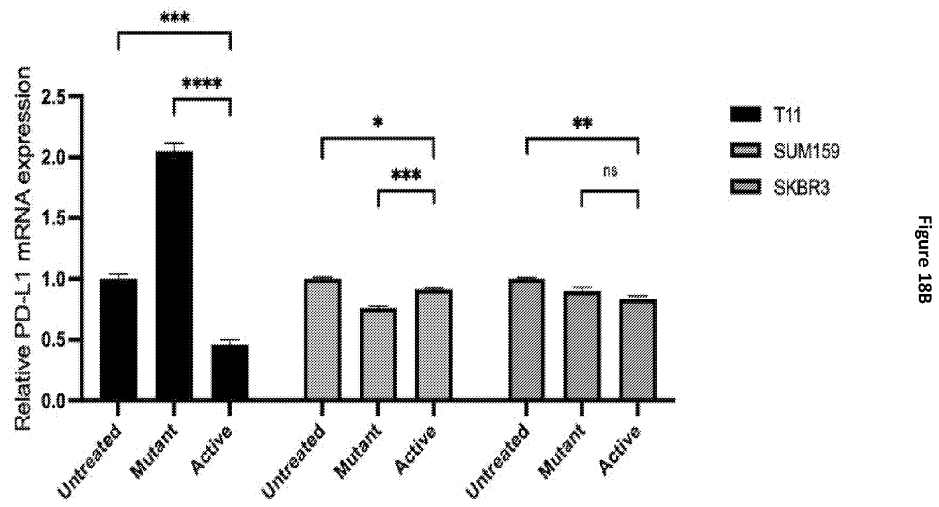

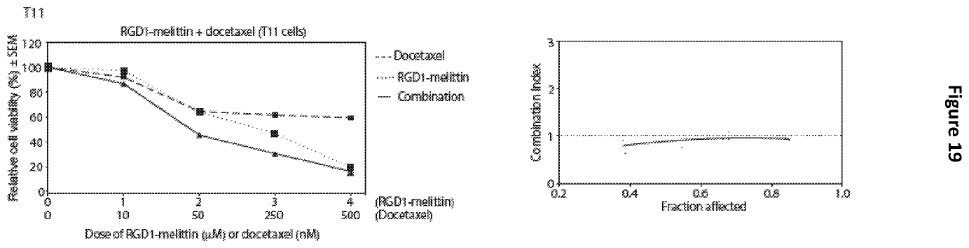

The results are presented in FIG. 18. In FIG. 18A, the Hoechst 33258-stained cell nuclei are presented in blue, and positively stained cells for Ki-67 and PD-L1 are presented in green respectively. The results showed that RGD1-melittin (but not inactive RGD1-melittin) reduced cell proliferation and PD-L1 expression in all three cell lines (T11, SUM159, and SKBR3).

The level of PD-L1 expression was also measured in T11, SUM159 and SKBR3 treated with active RGD1-melittin and mutant RGD1-melittin peptides, or vehicle treated only, using qRT-PCR.

Cells were seeded overnight at a density of 3×105 in a p6-well plate. The following day, cells were treated with RGD1-melittin and mutant RGD1-melittin for 24 h at IC50 concentrations. Cells were washed once with PBS before RNA was extracted from the cells using QIAzol Lysis. Reagent (QIAGEN) according to manufacturer's instructions. 500 ng of purified total RNA was used as the template for cDNA synthesis using the High Capacity cDNA Reverse Transcription Kit (Applied Biosystems). Relative quantification of PD-L1 transcript expression was obtained by quantitative real-time PCR (qRT-PCR) using fluorescent TaqMan probes (Applied Biosystems) and run on the ViiA 7TM Real-Time PCR machine (Applied Biosystems). Data were analysed by QuantStudio Real-Time PCR Software (Applied Biosystems) and results were expressed as fold change compared to untreated cells, respectively, after normalization against the GAPDH mRNA levels.

The results are presented in FIG. 18B. Active RGD1-melittin was found to significantly reduce the level of PD-L1 in each of the three cell lines (p<0.005 for the T11 cell line, p<0.05 for the SUM159 cell line and p<0.005 for the SKBR3 cell line).

### Example 11

Cell viability assays of T11, OVCAR3, SUM159 and SKBR3 cells treated with RGD1-melittin alone and in combination with docetaxel were performed. Cells were seeded overnight at a density of 5000 cells/p96-well plate and treated with active RGD1-melittin alone or combined with docetaxel and administered at concentrations indicated in a non-constant ratio in the respective cell lines for 24 h. Cell viability was assessed using the CellTiter-Glo® assay.

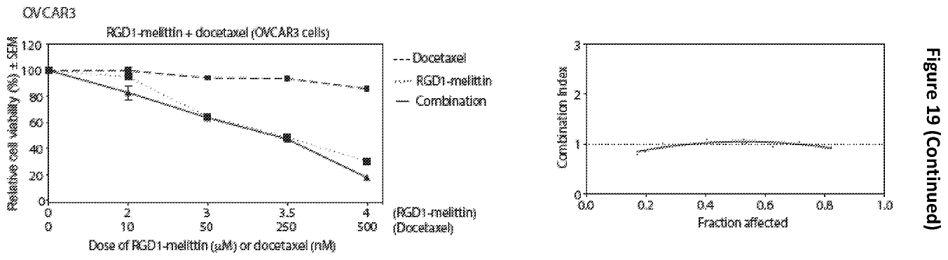

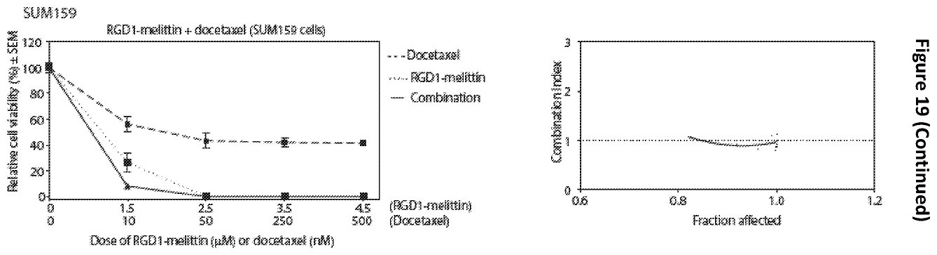

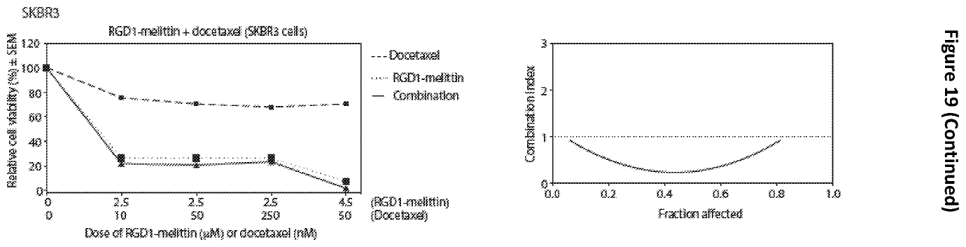

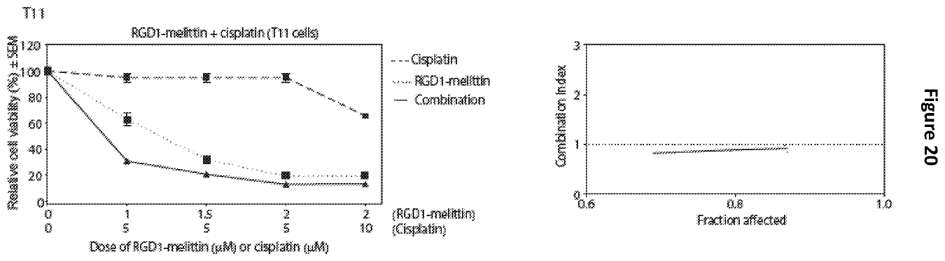

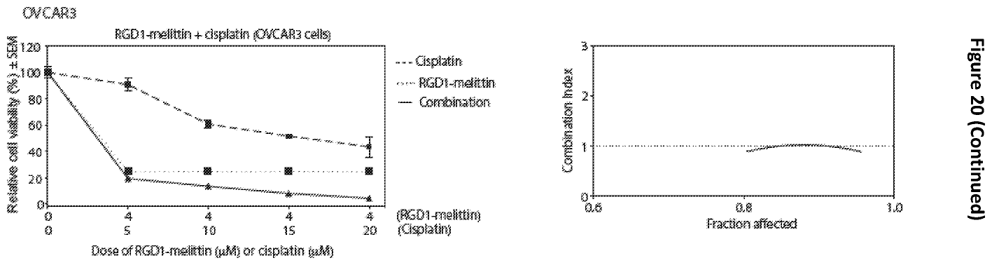

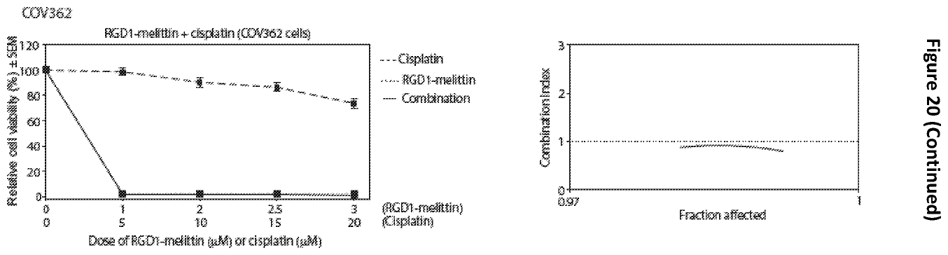

The results are presented in FIG. 19. The combination effect for different fractions of cells impacted in each combination were calculated using the Chou and Talalay algorithm included in the freely available CompuSyn software. Combination index (CI)<1 means synergism, CI=1 means additive and CI>1 means antagonism. The results show that RGD1-melittin significantly sensitized all four tested cell lines to docetaxel.

### Example 12

Cell viability assays of T11, OVCAR3, and COV362 cells treated with RGD1-melittin alone and in combination with cisplatin were performed. Cells were seeded overnight at a density of 5000 cells/p96-well plate and treated with active RGD1-melittin alone or combined with docetaxel and administered at concentrations indicated in a non-constant ratio in the respective cell lines for 24 h. Cell viability was assessed using the CellTiter-Glo® assay.

The results are presented in FIG. 19. The combination effect for different fractions of cells impacted in each combination were calculated using the Chou and Talalay algorithm included in the freely available CompuSyn software. Combination index (CI)<1 means synergism, CI=1 means additive and CI>1 means antagonism. The results show that RGD1-melittin significantly sensitized all three tested cell lines to cisplatin.

1. A fusion peptide comprising (1) SEQ ID NO: 1, or a variant thereof, and (2) an RGD-motif containing peptide, wherein the fusion peptide binds to the integrins αvβ6 and αvβ3.

2. (canceled)

3. The fusion peptide of claim 1 wherein the RGD-motif containing peptide comprises SEQ ID NO: 2 or a variant thereof.

4. The fusion peptide of claim 3 wherein the fusion peptide comprises SEQ ID NO: 3.

5. A fusion peptide comprising SEQ ID NO: 3, or a variant thereof.

6. A pharmaceutical composition comprising the fusion peptide of claim 1.

7. A method of treating cancer comprising the step of administering a therapeutically effective amount of the fusion peptide of claim 1 to a subject in need thereof.

8. The method of claim 7 wherein the cancer is a type of cancer wherein the integrins αvβ6 and/or αvβ3 are overexpressed on the plasma membrane of the tumour cell and/or associated vasculature.

9. A method of reducing tumour cell viability comprising the step of administering the fusion peptide of claim 1 in a therapeutically effective amount to a subject in need thereof.

10. The method of claim 7 additionally comprising the step of administering a therapeutically effective amount of the anticancer therapy to the subject.

11. A method of treating triple negative breast cancer, HER2 enriched breast cancer or ovarian cancer comprising the steps of: (1) administering a therapeutically effective amount of melittin; and (2) administering a therapeutically effective amount of docetaxel or cisplatin to a subject in need thereof.

12. (canceled)

13. (canceled)

14. A kit for treating cancer in a subject comprising the fusion peptide of claim 1, and together with instructions as to how to use the fusion peptide.

15. A nucleic acid molecule encoding the fusion peptide of claim 1.

16. (canceled)

17. A method of inhibiting PD-L1 expression in a subject suffering from cancer comprising the step of administering a therapeutically effective amount of the fusion peptide of claim 1 to a subject in need thereof.

18. (canceled)

19. (canceled)

20. A method of facilitating the cell penetration of an anticancer therapy in a subject suffering from cancer, comprising the step of administering a therapeutically effective amount of a combination therapy comprising administering: (1) a fusion peptide of claim 1; and (2) an anticancer therapy to the subject.

21. (canceled)

22. A method of treating cancer comprising the step of administering a therapeutically-effective amount of the pharmaceutical composition of claim 6 to a subject in need thereof.

23. A method of reducing tumour cell viability comprising the step of administering the pharmaceutical composition of claim 6 in a therapeutically effective amount to a subject in need thereof.


SEQUENCE LISTING (10 sequences)

SEQ ID NO: 1 - protein, 26 aa, Apis mellifera
GIGAVLKVLTTGLPALISWIKRKRQQ

SEQ ID NO: 2 - protein, 11 aa, Artificial Sequence
  RGD motif peptide
HGRGDLGRLKK

SEQ ID NO: 3 - protein, 37 aa, Artificial Sequence
  Fusion protein
HGRGDLGRLKKGIGAVLKVLTTGLPALISWIKRKRQQ

SEQ ID NO: 4 - protein, 4 aa, Apis mellifera
KRKR

SEQ ID NO: 5 - protein, 6 aa, Artificial Sequence
  Positive charge peptide
KKKRKV

SEQ ID NO: 6 - protein, 26 aa, Artificial Sequence
  Negative charge peptide
GIGAVLKVLTTGLPALISWIDEDEQQ

SEQ ID NO: 7 - DNA, 111 bp, Artificial Sequence
  Fusion protein coding sequence
cacggcaggggcgacctgggcaggctgaagaagggcatcggcgccgtgctgaaggtgctgaccaccggcctgcccgccctgatcagctggatcaagaggaagaggcagcag

SEQ ID NO: 8 - DNA, 111 bp, Artificial Sequence
  Fusion protein coding sequence
catggccgcggcgatctgggccgcctgaaaaaaggcattggcgcggtgctgaaagtgctgaccaccggcctgccggcgctgattagctggattaaacgcaaacgccagcag

SEQ ID NO: 9 - protein, 26 aa, Artificial Sequence
  Fusion protein
GIGAVLKVLTTGLPALISWIKKKRKV

SEQ ID NO: 10 - protein, 32 aa, Artificial Sequence
  Fusion protein
GIGAVLKVLTTGLPALISWIGRKKRRQRRRPQ